(19) United States
(12) Patent Application Publication
(10) Pub. No.: US 2025/0186713 A1
(43) Pub. Date: Jun. 12, 2025

(54) DISPOSABLE CARTRIDGES FOR ELECTROSTATIC APPLICATOR, SYSTEMS, AND METHODS THEREOF

(71) Applicant: Octet Medical, Inc., San Diego, CA (US)

(72) Inventors: Clifford A. Wright, San Diego, CA (US); Kyle Elsabee, San Diego, CA (US); Ron Lawrence, San Diego, CA (US); Michael Williams, San Diego, CA (US); Tom Lorenzana, San Diego, CA (US)

(73) Assignee: Octet Medical, Inc., San Diego, CA (US)

(21) Appl. No.: 18/930,826

(22) Filed: Oct. 29, 2024

Related U.S. Application Data
(60) Provisional application No. 63/607,153, filed on Dec. 7, 2023.

(51) Int. Cl. A61M 11/02

(52) U.S. Cl. CPC A61M 11/02; A61M 2205/121; A61M 2205/123

(57) ABSTRACT

A disposable fluid delivery system for an electrostatic applicator. The system includes a nozzle cap including a distal end including a delivery outlet and an open proximal end. A nozzle housing can be included with a proximal inlet end and an open distal end connected to the open proximal end of the nozzle cap. A conductive insert is at least partially positioned between the open distal end and the proximal inlet end and includes a portion of a flow pathway formed at least partially between the nozzle cap, the nozzle housing, and the conductive insert. A high voltage connector is extended at least partially through the nozzle housing and electrically connected to the conductive insert and configured to be in electrical communication with a high voltage module and electrostatically charge fluid contents within the flow pathway.

### CROSS-REFERENCE TO RELATED APPLICATIONS

This application claims priority to, and the benefit of, U.S. Provisional Patent Application Ser. No. 63/607,153, filed on Dec. 7, 2023, entitled “DISPOSABLE CARTRIDGES FOR ELECTROSTATIC APPLICATOR, SYSTEMS, AND METHODS THEREOF”, the disclosure of which is hereby incorporated by reference in their entirety.

### FIELD

The solution of this disclosure relates to devices, systems, and methods for applying one or more medicaments (e.g., one or more biologics, polymer spun wound dressing, antiseptics, or anesthetic) to a treatment site (e.g., a wound surface of subject). More specifically, the devices, systems, and methods are directed towards electrostatic applicator devices with a disposable cartridge for housing a variety of solutions.

### BACKGROUND

Infectious disease is too often acquired in places that should be safe, such as ambulances, hospitals, clinical settings, and other areas such as assisted living facilities. Indeed, these health care associated infections (HAI) pose a major threat to patient safety and cause an unnecessary financial burden. Surgical site infections, for example, which are a large contributor to HAIs, can create pain and discomfort for a patient but also can contribute to longer and/or repeat hospital admissions. Numerous antibacterial and/or analgesic compounds are available to help treat the patient and avoid infections; however, despite the prevalence of these compounds, current delivery methods can often be less than efficacious.

Oral and intravenous administration, for example, are often insufficient to effectively control severe pain at or treat a specific region of the human body, and giving a high concentration dose may lead to adverse events. To overcome some of the issues related to oral and intravenous administration, delivery vehicles such as hydrogels have been developed to provide spatial and temporal control over the release of various therapeutic agents, including small molecule drugs, peptides, and cells. However, hydrogels can also have certain undesirable characteristics, including being expensive and difficult to sterilize.

More recently, electrospraying has emerged as a technology with potential biomedical applications. Electrospraying includes providing an electrostatic charge to a fluid as the fluid is expelled from an electrostatic sprayer. The electric field can cause the expelled liquid to break up into diminutive droplets, e.g., on the order of microns, which can bind to a treatment site relatively evenly and less solution. That said, not all solutions, including antiseptics and/or analgesics, respond equally to the same electrostatic conditions-differences in the viscosity and/or dielectric properties of the solution can require different optimal configurations for an electrostatic sprayer device. Current electrostatic sprayer devices do not provide modular systems that can account for various types of different solutions within one electrostatic sprayer device.

This disclosure resolves these and other issues of the art.

### SUMMARY

The subject of this disclosure is an electrostatic applicator for emitting contents from a disposable cartridge (e.g., treatment solution contained in the cartridge) to a treatment site of a patient.

In some examples, a disposable fluid delivery system is disclosed for an electrostatic applicator. The system includes a nozzle cap including a distal end including a delivery outlet and an open proximal end. A nozzle housing can be included with a proximal inlet end and an open distal end connected to the open proximal end of the nozzle cap. A conductive insert is at least partially positioned between the open distal end and the proximal inlet end and includes a portion of a flow pathway formed at least partially between the nozzle cap, the nozzle housing, and the conductive insert. A high voltage connector is extended at least partially through the nozzle housing and electrically connected to the conductive insert and configured to be in electrical communication with a high voltage module and electrostatically charge fluid contents within the flow pathway.

In some examples, the high voltage connector includes a first segment orthogonal relative to a second segment. At least one of the first and second segments includes an outward flexed contact configured to electrically connect with a high voltage contact of the electrostatic applicator.

In some examples, the high voltage connector includes a first segment angled relative to a second segment. At least one of the first and second segments includes an outward flexed contact configured to electrically connect with a high voltage contact of the electrostatic applicator.

In some examples, the high voltage connector includes a spring (e.g., a folded leaf spring, a return spring, a compression spring, a coil spring, a tension spring, pre-loaded resilient material, etc.). In some examples, the high voltage connector is formed of a conductive material with one or more resilient aspects.

In some examples, the nozzle housing is hermetically sealed to the nozzle cap forming an atomizing air chamber.

In some examples, the system includes a nozzle tip positioned between the nozzle cap and the nozzle housing, the nozzle tip including a distal end aligned with the delivery outlet and a proximal end aligned and in fluid communication with the flow pathway of a distal end of the conductive insert. Air from the atomizing air chamber is able to flow around the nozzle tip and egress out of the delivery outlet to atomize fluid egressing from the flow pathway.

In some examples, the atomizing air chamber includes a volume of at least approximately 900 mm³.

In some examples, the atomizing air chamber includes a volume of at least approximately 100 mm³.

In some examples, the atomizing air chamber includes a volume of between at least approximately 100 mm³ and approximately 1,000 mm³.

In some examples, the nozzle housing includes an air supply port in fluid communication with the atomizing air chamber for atomizing electrostatically charged fluid from the nozzle cap.

In some examples, the nozzle cap includes a plurality of holes on a face of the distal end and selectively arranged around the delivery outlet, the plurality of holes in fluid communication with the atomizing air chamber.

In some examples, the system includes a delivery tube at least partially extended from the delivery outlet and in fluid communication with the flow pathway.

In some examples, the face of the distal end is curved inwardly or curved outwardly.

In some examples, the nozzle cap is configured so that during use, air pumped from the atomizing air chamber through and out through the plurality of holes creates an air curtain around the electrostatically charged fluid contents egressing from the delivery outlet.

In some examples, the conductive insert is nested in a cavity of the nozzle housing positioned between the open distal end and the proximal inlet.

In some examples, the system includes a syringe removably connected to the proximal inlet end of the nozzle housing, the syringe including a barrel portion and a plunger configured to advance fluids from within the barrel portion and through the flow pathway. A cartridge housing at least partially enclosing the nozzle housing, the nozzle cap, the high voltage connector, and the syringe.

In some examples, the high voltage connector is a spring extended at least partially through a high voltage port of the nozzle housing and includes a high voltage contact segment extended at least partially through an aperture of the cartridge housing.

In some examples, a contact of the high voltage connector to provide a voltage potential of approximately 1 V to approximately 40 kV to the conductive insert.

In some examples, a contact of the high voltage connector is in physical contact with the conductive insert to provide a voltage potential of approximately 1 V to approximately 40 kV to fluid in the flow pathway.

In some examples, the cartridge housing is a moldable plastic material, wherein the cartridge housing includes a plurality of moldable plastic connectable sections.

In some examples, the syringe contains contents including one or more of an antiseptic, a disinfectant solution, an analgesic, an exosome, a biologic, and/or a liquid bandage solution.

In some examples, the system includes a locking mechanism between a distal end of the syringe and the proximal inlet end of the nozzle housing, the locking mechanism including at least one retainer tab configured to securely connect with the distal end of the syringe and at least one snap connector configured to releasably snap onto the nozzle housing, wherein the locking mechanism comprises a portion of the flow pathway.

In some examples, the system includes the portion of the flow pathway of the locking mechanism includes an openable seal.

In some examples, the system includes the at least one retainer tab is configured prevent the syringe from being removed from the disposable fluid delivery system.

In some examples, the system includes an outlet diameter of the delivery outlet ranges between approximately a 16-gauge to 25-gauge tube size.

In some examples, the system includes a reusable electrostatic applicator including a cartridge chamber sized and shaped to accept the cartridge housing. The applicator can include a high voltage module configured to be in electrical communication with an at least partially moveable voltage contact of the high voltage connector. A piston positioned proximate the cartridge chamber and configured to advance the plunger enclosed in the cartridge housing when the cartridge housing is assembled with the cartridge chamber.

In some examples, the reusable electrostatic applicator includes a motor configured to move the piston, one or more processors, and memory storing instructions that, when executed by the one or more processors, causes the reusable electrostatic applicator to receive an activation signal; output a control signal to the motor to actuate the piston; and output a control signal to a switch to provide voltage from the high voltage module to the high voltage connector.

In some examples, the reusable electrostatic applicator includes a housing base including a voltage source, a device housing including the cartridge chamber, and a handle extending between the housing base and the device housing.

In some examples, an applicator system is disclosed for delivering a treatment solution to a target site. A portable reusable electrostatic applicator including a device housing configured to be handheld, a motor in the device housing configured to drive a piston, a voltage source in the device housing, a high voltage module electrically connected to the voltage source, a cartridge chamber, and a disposable cartridge removably insertable in the cartridge chamber. The disposable cartridge includes a nozzle cap including a distal end including a delivery outlet and a proximal end. A nozzle housing can include a proximal inlet end and an open distal end connected with the open proximal end of the nozzle cap. A conductive insert is between the open distal end and the proximal inlet end and including a portion of a flow pathway formed at least partially between the nozzle cap, the nozzle housing, and the conductive insert. A high voltage connector is extended at least partially through the nozzle housing and electrically connected to the conductive insert and in electrical communication with the high voltage module. A syringe including a barrel portion and a plunger is configured to advance fluids from within the barrel portion and through the flow pathway. A cartridge housing is included at least partially enclosing the nozzle cap, the nozzle housing, the high voltage connector, and the syringe.

In some examples, the cartridge chamber includes a wall including a high voltage contact in electrical communication with the high voltage module and an air supply port in fluid communication with a pump positioned in the device housing. The high voltage connector is in electrical communication with the high voltage contact of the wall. The contact of the voltage wire is in physical contact with an outer surface of the conductive insert to provide a voltage potential of approximately 1 V to approximately 40 kV.

In some examples, the reusable electrostatic applicator includes one or more processors; and memory storing instructions that, when executed by the one or more processors, causes the reusable electrostatic applicator to receive an activation signal; and output a control signal to a motor controlling (a) a voltage potential from the high voltage module to the conductive insert via the high voltage connector, (b) a position of the piston and the plunger of the disposable cartridge, and/or (c) a pump regulating air flow from the device housing into the air supply port.

In some examples, the disposable cartridge includes an integrated memory including information related to one or more operational parameters of contents stored within the syringe and/or another fluid reservoir of the disposable cartridge, wherein the one or more processors of the reusable electrostatic applicator are configured to communicate with the integrated memory to retrieve information related to the contents of the disposable cartridge and control at least one of a flow rate, a voltage potential, and a nozzle setting.

In some examples, the system includes an accelerometer configured to output a movement signal to the one or more processors in response to detecting movement of the electrostatic applicator system. A display screen on the electrostatic applicator system is activated by a wake signal from the one or more processors in response to the one or more processors receiving the movement signal; and wherein the activation signal is a user input into the display screen.

In some examples, the high voltage connector includes a first segment orthogonal relative to a second segment. At least one of the first and second segments includes an outward flexed contact configured to electrically connect with a high voltage contact of the electrostatic applicator.

In some examples, the high voltage connector includes a first segment angled relative to a second segment, wherein at least one of the first and second segments includes an outward flexed contact configured to electrically connect with a high voltage contact of the electrostatic applicator.

In some examples, an atomizing air chamber is formed between the nozzle cap and the nozzle housing.

In some examples, a nozzle tip is positioned between the nozzle cap and the nozzle housing, the nozzle tip including a distal end aligned with the delivery outlet and a proximal end aligned and in fluid communication with the flow pathway of a distal end of the conductive insert. Air from the atomizing air chamber is able to flow around the nozzle tip and egress out of the delivery outlet to atomize fluid egressing from the flow pathway.

In some examples, the atomizing air chamber includes a volume of at least approximately 900 mm³.

In some examples, the atomizing air chamber includes a volume of at least approximately 100 mm³.

In some examples, the atomizing air chamber includes a volume of between at least approximately 100 mm³ and approximately 1,000 mm³.

In some examples, the nozzle housing includes an air supply port in fluid communication with the atomizing air chamber for atomizing electrostatically charged fluid from the nozzle cap.

In some examples, the nozzle cap includes a plurality of holes on a face of the distal end and selectively arranged around the delivery outlet, the plurality of holes in fluid communication with the atomizing air chamber.

In some examples, a delivery tube is at least partially extended from the delivery outlet and in fluid communication with the flow pathway.

In some examples, the face of the distal end is curved inwardly or curved outwardly.

In some examples, the nozzle cap is configured so that during use, air pumped from the atomizing air chamber through and out through the plurality of holes creates an air curtain around the electrostatically charged fluid contents egressing from the delivery outlet.

In some examples, the conductive insert is nested in a cavity of the nozzle housing positioned between the open distal end and the proximal inlet.

In some examples, the high voltage connector is a spring extended at least partially through a high voltage port of the nozzle housing and includes a high voltage contact segment extended at least partially through an aperture of the cartridge housing.

In some examples, a contact of the high voltage connector to provide a voltage potential of approximately 1 V to approximately 40 kV to the conductive insert.

In some examples, a contact of the high voltage connector is in physical contact with the conductive insert to provide a voltage potential of approximately 1 V to approximately 40 kV to fluid in the flow pathway.

In some examples, the cartridge housing is a moldable plastic material, wherein the cartridge housing includes a plurality of moldable plastic connectable sections.

In some examples, the syringe contains contents including one or more of an antiseptic, a disinfectant solution, an analgesic, an exosome, a biologic, and/or a liquid bandage solution.

In some examples, a locking mechanism is between a distal end of the syringe and the proximal inlet end of the nozzle housing, the locking mechanism including at least one retainer tab configured to securely connect with the distal end of the syringe and at least one snap connector configured to releasably snap onto the nozzle housing, wherein the locking mechanism comprises a portion of the flow pathway, wherein the portion of the flow pathway of the locking mechanism includes an openable seal.

In some examples, the at least one retainer tab is configured prevent the syringe from being removed from the disposable fluid delivery system.

In some examples, an outlet diameter of the delivery outlet ranges between approximately a 16-gauge to 25-gauge tube size.

In some examples, a method for operating an electrostatic applicator system is disclosed. The method includes inserting a first disposable cartridge into a chamber housing of the electrostatic applicator system such that (a) a voltage contact of a high voltage connector at least partially within a nozzle housing and at least partially extended through a cartridge housing of the first disposable cartridge contacts a voltage contact of the electrostatic applicator system; (b) an air supply port of the nozzle housing of the first disposable cartridge fluidly connects with an air supply of the electrostatic applicator system; and (c) a syringe of the first disposable cartridge aligns with a piston of the electrostatic applicator system, the syringe containing a first fluid. The method can include causing, by an activation input to the electrostatic applicator system, a voltage potential to be delivered by the high voltage connector to a conductive insert of the first disposable cartridge and electrostatically charge fluid contents within a flow pathway.

In some examples, the method includes forming an atomizing air chamber between a nozzle cap and a nozzle housing of the first disposable cartridge to atomize electrostatically charged fluid contents from the flow pathway and egressing through a delivery outlet of the nozzle cap.

In some examples, the method includes directing air, from a pump of the electrostatic applicator system, through an air supply port of the nozzle housing and into the atomizing air chamber; and causing, by moving the plunger, the electrostatically charged fluid contents from the syringe to delivered through a delivery outlet of the nozzle cap.

In some examples, the first disposable cartridge includes an integrated memory including information related to operational parameters of the first fluid, and wherein inserting the first disposable cartridge into the chamber housing causes the operational parameters to be transmitted to a memory of electrostatic applicator system, the operational parameters including at least one of a speed of the motor, an air intake, and a voltage applied to a flow pathway of the respective disposable cartridge.

To the accomplishment of the foregoing and related ends, certain illustrative aspects are described herein in connection with the following description and the appended drawings. These aspects are indicative, however, of but a few of the various ways in which the principles of the claimed subject matter may be employed and the claimed subject matter is intended to include all such aspects and their equivalents. Other advantages and novel features may become apparent from the following detailed description when considered in conjunction with the drawings.

### BRIEF DESCRIPTION OF THE DRAWINGS

The above and further aspects of this invention are further discussed with reference to the following description in conjunction with the accompanying drawings, in which like numerals indicate like structural elements and features in various figures. The drawings are not necessarily to scale, emphasis instead being placed upon illustrating principles of the invention. The figures depict one or more implementations of the inventive devices, by way of example only, not by way of limitation.

FIG. 1A depicts a perspective view of an example electrostatic applicator with a disposable cartridge in an exploded state.

FIG. 1B depicts a perspective view of the example electrostatic applicator of FIG. 1A with the disposable cartridge in an assembled state.

FIG. 2A depicts a forward perspective view of the example cartridge of FIGS. 1A to 1B.

FIG. 2B depicts a rear perspective view of the example cartridge of FIGS. 1A to 1B.

FIG. 3A depicts a lower perspective view of the example cartridge of FIGS. 1A to 2B.

FIG. 3B depicts a top plan view of the example cartridge of FIGS. 1A to 2B.

FIG. 4A depicts a bottom plan view of the example cartridge of FIGS. 1A to 2B.

FIG. 4B depicts a side plan view of the example cartridge of FIGS. 1A to 2B.

FIG. 5A depicts a cross-section view of section 5A-5A from FIG. 4A showing aspects of the example cartridge of FIGS. 1A to 2B.

FIG. 5B depicts a perspective view of exemplary aspects of components of the example cartridge of FIGS. 1A to 2B in an assembled state.

FIG. 6A depicts a perspective view of exemplary aspects of components of the example cartridge of FIGS. 1A to 2B in an exploded state.

FIG. 6B depicts a side view of exemplary aspects of components of the example cartridge of FIGS. 1A to 2B in an exploded state.

FIG. 7A depicts a close-up, side cross-section view showing aspects of the example cartridge of FIGS. 1A to 2B in an assembled state.

FIG. 7B depicts a close-up, top cross-section view showing aspects of the example cartridge of FIGS. 1A to 2B in an assembled, operational state.

FIG. 8A depicts a close-up, side cross-section view showing aspects of an example locking mechanism.

FIG. 8B depicts a forward perspective view of the locking mechanism of FIG. 8A.

FIG. 8C depicts a rear perspective view of the locking mechanism of FIG. 8A.

FIG. 9A depicts a perspective view of an exemplary nozzle tip.

FIG. 9B depicts a side-cross section view of the exemplary nozzle tip of FIG. 9A.

FIG. 9C depicts a side-cross section view of another exemplary nozzle tip.

FIG. 9D depicts a side-cross section view of another exemplary nozzle tip.

FIG. 9E depicts a side-cross section view of another exemplary nozzle tip.

FIG. 10A depicts a forward perspective view of assembled components of an example electrospinning cartridge contemplated for use with the example electrostatic applicator and related cartridge housing of FIGS. 1A to 4B.

FIG. 10B depicts a side view of exemplary aspects of the components of the example of FIG. 10A in an exploded state.

FIG. 11 depicts a close-up, top cross-section view showing aspects of the example assembled components of FIGS. 10A to 10B in an assembled, operational state.

FIG. 12 is a flow diagram for operating an example applicator and disposable cartridge system, according to the present disclosure.

FIG. 13 is a computer architecture diagram showing a general computing system for implementing aspects of the present disclosure in accordance with one or more embodiments described herein.

### DETAILED DESCRIPTION

Although example embodiments of the disclosed technology are explained in detail herein, it is to be understood that other embodiments are contemplated. Accordingly, it is not intended that the disclosed technology be limited in its scope to the details of construction and arrangement of components set forth in the following description or illustrated in the drawings. The disclosed technology is capable of other embodiments and of being practiced or carried out in various ways.

It must also be noted that, as used in the specification and the appended claims, the singular forms “a,” “an” and “the” include plural referents unless the context clearly dictates otherwise. By “comprising” or “containing” or “including” it is meant that at least the named compound, element, particle, or method step is present in the composition or article or method, but does not exclude the presence of other compounds, materials, particles, method steps, even if the other such compounds, material, particles, method steps have the same function as what is named.

Relative terms, such as “about,” “substantially,” or “approximately” are used to include small variations with specific numerical values (e.g., +/−x %,), as well as including the situation of no variation (+/−0%). In some examples, the numerical value x is less than or equal to 10—e.g., less than or equal to 5, to 2, to 1, or smaller.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the disclosed technology. Similarly, it is also to be understood that the mention of one or more components in a device or system does not preclude the presence of additional components or intervening components between those components expressly identified.

As discussed herein, a treatment site of a “subject” or “patient” may be a wound site or treatment of a human or any animal. It should be appreciated that an animal may be a variety of any applicable type, including, but not limited thereto, mammal, veterinarian animal, livestock animal or pet type animal, etc. As an example, the animal may be a laboratory animal specifically selected to have certain characteristics similar to a human (e.g., rat, dog, pig, monkey, or the like). It should be appreciated that the subject may be any applicable human patient, for example.

As discussed herein, “operator” may include, but is not limited to, a doctor, surgeon, nurse, physical therapist, or other healthcare professional, or any other suitable individual, or delivery instrumentation associated with the application of a treatment solution of a treatment site of a subject.

As discussed herein, “treatment solution” may be one or more fluids (e.g., liquid and/or emulsion solution, gels, and/or mixtures) and may include one or more of an antiseptic solution, a disinfectant solution, an analgesic, an exosome, a biologic, chlorohexidine gluconate, povidone-iodine, and/or a liquid bandage solution. The analgesic can include one or more of lidocaine, levobupivacaine, acemetacin, ketorolac, and ceftazidime. The biologic can include one or more of stem cells and/or mammalian primary cells, medicaments, gels (e.g., hydrogels), reconstitutable aspects (e.g., immiscible and/or lyophilized ingredients mixable with one or more solvents). The disinfectant can include one or more alcohols, aldehydes, oxidatives, phenols, quaternary ammonium compounds, etc., antibacterial agents, biguanides, analgesic agents, surfactant agents, and/or debridement components or any other contents and/or medication contemplated for storage in a cartridge of this disclosure that can be delivered (e.g., applied, deposited, and/or sprayed by an electrostatic applicator) to a treatment site of a patient. The treatment solution can include any concentration or mixture of herein disclosed ingredients.

The term “treatment solution” can also include one or more tracking materials (e.g., gels with tracking aspects, intermixed with the treatment solution). The treatment solution can include any number of small molecule drugs, peptides, cells, and other therapeutics. In some aspects, the treatment solution can include one or more active pharmaceutical ingredients, growth factors, trophic factors, exosomes, mammalian regenerative cells, and/or a supportive matrix. In some aspects, the treatment solution can include lidocaine, levobupivacaine, acemetacin, ketorolac, and or the like or any combination thereof.

The terms “distal” or “proximal” are used in the following description with respect to a position or direction relative to a reference point (e.g., such as a user [e.g., the treating physician or medical interventionalist]). “Distal” or “distally” are a position distant from or in a direction away from the reference point. “Proximal” or “proximally” or “proximate” are a position near or in a direction toward the reference point.

An increasing amount of care and resources has been focused on creating effective treatments for pain and infection care. The most common method of treating pain and infection includes oral and intravenous (IV) administration of drugs. Although these methods are prevalent, oftentimes the efficacy of the drug delivery method is lacking. For example, when drugs are administered orally or via IV methods, their treatment effect is generalized and not localized—the drug targets the entire patient instead of a localized treatment site. To overcome some of these issues, delivery vehicles such as topical treatments and, more particularly, hydrogel compounds have been developed to provide spatial and temporal control over the release of various therapeutic agents, including small molecule drugs, peptides, and cells. However, these treatments can also have drawbacks. These often synthetic carriers of therapeutics can be costly and, since they are fragile polymer chains, it can be difficult to sterilize polymer chains of the solution/hydrogel combination.

More recently, the concept of electrospraying treatment solutions, including antibacterial and/or analgesic solutions, to treatment sites has been considered. Electrostatic spraying is a technique that subjects a treatment solution to an electric field to charge the fluid. The electric field, provided by a voltage source, can create a charge to the fluid being administered (e.g., a positive charge or a negative charge). This is particularly helpful in the biomedical arena since the natural resting state of human cells is negative (i.e., a state of negative charge). This imbalance is created by potassium and sodium ions inside and outside the cell that establishes electrical capacity in a patient's body. This polarity differential creates a natural attraction between the treatment site and the solution being sprayed.

However, the negative/positive attraction is not the only benefit of electrostatic spraying. For example, subjecting the fluid to this electrical field can also produce diminutive droplets (e.g., micron sizes), providing a relatively uniformly distributed layer of treatment solution. When the electrical stresses due to the charge builds in a liquid droplet beyond its surface tension, the droplet disintegrates and/or atomizes into very fine droplets-which is known as Rayleigh disintegration or coulomb fission. As discussed herein, the term “atomize” is understood as some or all of the process of converting a substantially liquid solution into very fine particles or droplets. The solvent dielectric constant or conductivity can play a crucial role in dictating particulate morphology. Other factors that affect the way a liquid atomizes include vapor pressure, viscosity and miscibility of the treatment solution, voltage applied to the solution, etc.

It is noted that prior designs for electrosprayer devices did not take these various types of parameters into consideration. This is because most prior art electrostatic device focused on spraying one type of solution-consider the common examples of ink jet sprayers, paint sprayers, etc., which sprayed consistent solutions at consistent flow rates and with consistent voltage potentials. Further, these types of applications did not focus on operating parameters. The present disclosure provides solutions that can maintain sterility for each administration of a treatment solution by providing individualized, pre-filled disposable cartridges housing the components used for electrospraying. Further, each disposable cartridge can be individually tailored and/or communicate with a reusable electrostatic applicator to individually tailor the parameters needed to administer the preferred particle (e.g., nano to microparticles) droplets for targeted treatment.

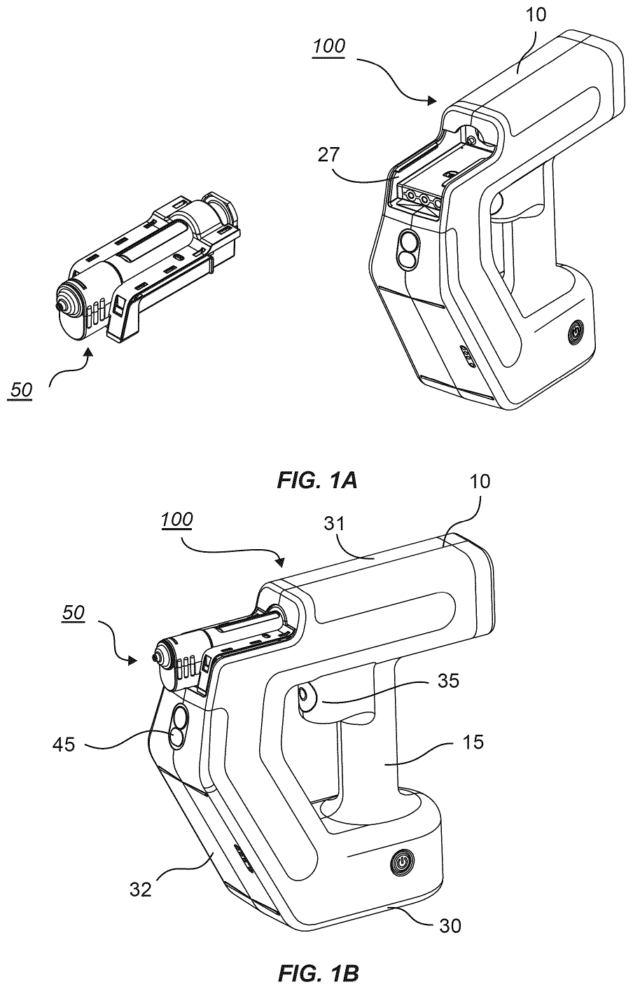

Turning to the drawings, FIG. 1A provides an example applicator 100 positioned in an exploded state with an example disposable cartridge 50. Applicator 100 can include an applicator housing 10 which can include an upper portion 31, a lower base portion 30 at the lower section, and a handle portion 15 between portions 30 and 31. A forward cartridge support portion 32 can also be positioned between portions 30 and 31. In some aspects, an obtuse angle can be formed between aspects of outer surfaces of portions 30 and 32 while handle portion 15 can be orthogonal to portions 30 and/or 31. While applicator housing 10 is shown as a handheld pistol-shaped housing, nothing requires the reusable applicator 100 to have a pistol-shaped design, as the components herein can also be combined in other electrosprayer designs, for example and not limited to a fully-cylindrical, handheld electrosprayer design. Other shapes and configurations for applicator 100 are also contemplated, as needed or required.

Portion 30 can house a battery B that provides the voltage potential to create the electrical field at the nozzle of cartridge 50 and/or can power the components of applicator 100 (e.g., CPU and/or a high-voltage (HV) module). Battery B can include one or more batteries, including, for example, direct current batteries such as lithium ion batteries. Battery B can provide sufficient voltage to create the voltage potential described above, including but not limited to approximately 1 V to approximately 40 kV. In some examples, the range can be approximately 1 V to 8 kV. In some examples, the voltage supply of battery B can be one or more rechargeable batteries. In this example, aspects of the applicator 100, such as portion 32, can engage with a charging base (not shown). Example components of applicator 100 (e.g., battery B, the HV module, an air pump, a circuit board, a target sensor 45, a piston to drive aspects of cartridge 50, a high voltage wall, one or more high voltage contacts, one or more air supply ports, a motor, a user interface, one or more processors such as a central processing unit (CPU)) as well as other internal components of the applicator 100 and the referenced charging base are described in U.S. application Ser. No. 18/110,854 filed Feb. 16, 2023, which is incorporated by reference in its entirety as if set forth verbatim herein.

In some examples, applicator 100 can include an actuator, e.g., a button 35 or trigger that can activate and/or initiate the components of applicator 100. For example, the button 35 can initiate power from the battery B. By activating and/or initiating power from battery B, the system is powered to electrostatically charge the liquid solution of the cartridge via direct charging, induction charging, indirect charging, or any combination thereof. In the case of direct charging, liquid solution of cartridge 50 can flow through an electrically conductive tube or other conductive aspects (e.g., conductive insert 120, described below) that is electrostatically charged such that the liquid solution flowing in a fluid pathway is contacted and charged by direct contact. In some examples, battery B can power the HV module, pump, circuit board, target sensor 45, motor, user interface, one or more processors of applicator 100 (e.g., central processing unit (CPU)) as well as components of cartridge 50. The CPU, for example, can facilitate activation of applicator 100, receiving and outputting signals relating to the voltage, flow rate, proximity, etc., for the particular liquid treatment solution, and the like. Other features can be included in the applicator 100 that can obviate the need for button 35, including, for example, accelerometers, activation inputs from a user device, etc.

In some aspects, the HV module can be configured to adjust or otherwise control operational aspects of cartridge 50 including but not limited to frequency, duty cycle, and input voltage to yield varying output voltages at different efficiencies. In some aspects, the HV module can be a closed loop system that monitors the output voltage and adjusts input parameters to optimize the output to a desired voltage for use with cartridge 50. The HV module can also be configured to produce positive and/or negative high voltage using the same board. In some aspects, the HV module can include a printed circuit board that is double sided so as to minimize footprint used within housing 10.

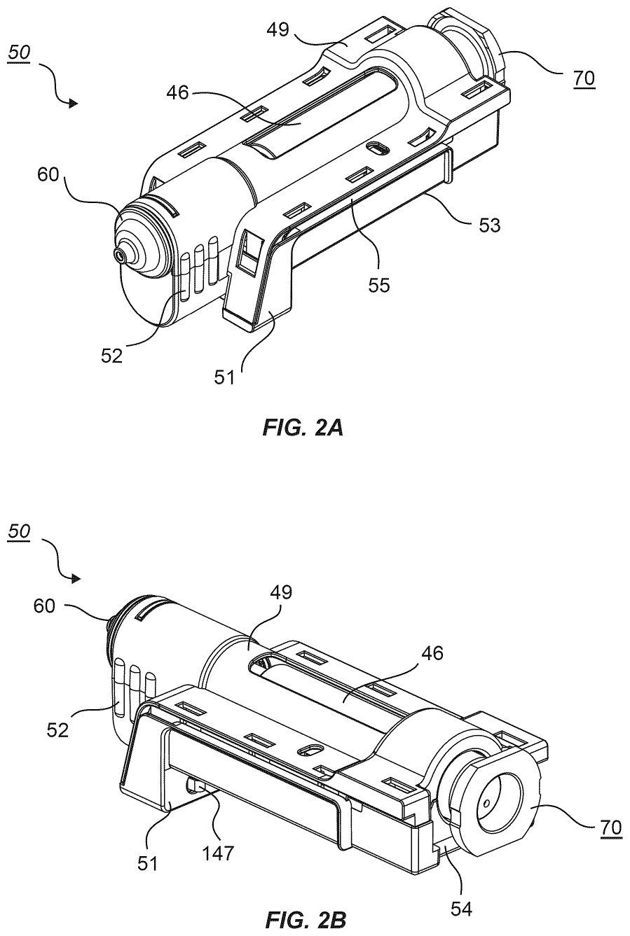

Applicator housing 10 can include a cartridge chamber 27 sized and positioned to accept a cartridge housing 49 of the disposable cartridge 50, as explained more particularly below in FIGS. 2A-4B. Cartridge chamber 27 can include a distal stepped portion through which connecting ports of internal components of housing 10 (e.g., applicator ports such as air supply ports, voltage ports, etc.) can connect to aspects of cartridge 50 (e.g., voltage contact(s), air supply port(s), cartridge retention mechanisms, etc.). Example cartridge retention mechanisms and related systems are described in U.S. application Ser. No. 18/235,324 filed Aug. 17, 2023, which is incorporated by reference in its entirety as if set forth verbatim herein. In some examples, cartridge 50 can include an NFC tag and/or other internal memory of cartridge 50 that can include information about contents stored therein (e.g., liquid treatment solution, recommended operational parameters, tracking information, expiry date, user information, manufacturer information, etc.).

In some aspects, this information of cartridge 50 can be used, for example, by applicator 100 to monitor the type of contents being delivered to a treatment site (e.g., pre-loaded contents of cartridge 50), its treatment solution volume as well as modify the applied voltage (e.g., amount, negative, positive, etc.), flow rate, recommended travel distance from the applicator 100 to the treatment site (i.e., proximity), etc., for the particular treatment solution. In some examples, this information can be stored on an integrated memory, which can include but is not limited to RAM, ROM, EPROM, EEPROM, etc. The information on any integrated memory can be relayed to the applicator 100 using the integrated memory and/or NFC tag, and this information can be used to adjust or otherwise control operational aspects of cartridge (e.g., components of syringe 70, flow rate, air intake from the air supply of the applicator 100, a voltage applied (e.g., applied by the high-voltage connector 147 to a portion of a flow pathway of cartridge 50)). In some aspects, one or more processors of the applicator 100 can be configured to read information of the NFC tag or other internal memory of cartridge 50 related to operational parameters of cartridge 50; and present the read information of the NFC in the display screen (e.g., information such as identification of contents of cartridge 50, volume information, etc.). In some aspects, operational information can be written to the NFC by the processor of the applicator 100.

Although the examples shown in FIGS. 1A-1B position the cartridge chamber 27 at the top of applicator 100, nothing requires the cartridge chamber 27 to be so located. For example, the cartridge chamber 27 can be positioned on the side of the applicator 100 (e.g., the side of the housing 10 of applicator 100, or any other location) as well as within an enclosed cavity of applicator 100. In some aspects, the HV module of applicator 100 can include electrical components for powering the components used for charging and spraying the liquid solution of cartridge 50. For example, the HV module can include the components used for applying the voltage to the conductive insert 120 of cartridge 50 so as to control the voltage applied to the liquid solution of cartridge 50, and/or for providing electricity to actuate a plunger 71 (or the motor for operating a syringe 70 including advancing plunger 71) so as to control the flow rate of liquid solution from cartridge 50 and through the nozzle. Within housing 10, a piston and corresponding motor can be positioned to actuate syringe 70 of cartridge 50. The piston can be positioned between the motor and an HV wall adjacent or part of chamber 27. On the opposite side of the HV wall, cartridge 50 can be positioned when attached to chamber 27 of housing 10. In some aspects, when cartridge 50 is positioned as in FIG. 1B, a plunger, piston, or other fluid advancing aspects within barrel 72 of syringe 70 of cartridge 50 can be advanced by the piston, through the HV wall, to advance contents of cartridge 50 through nozzle assembly.

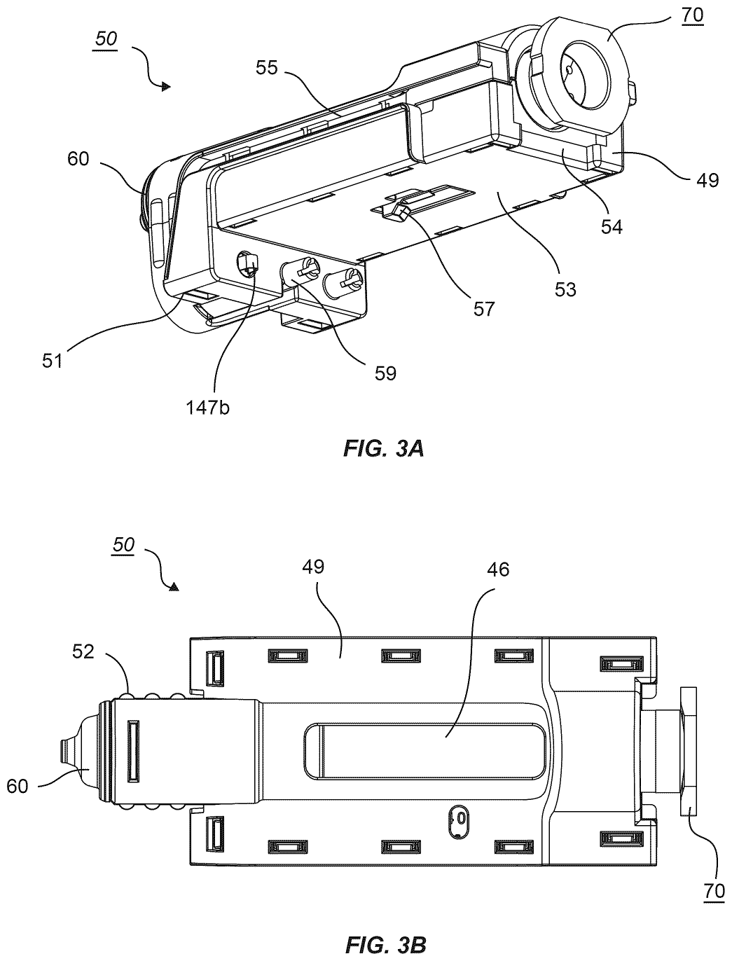

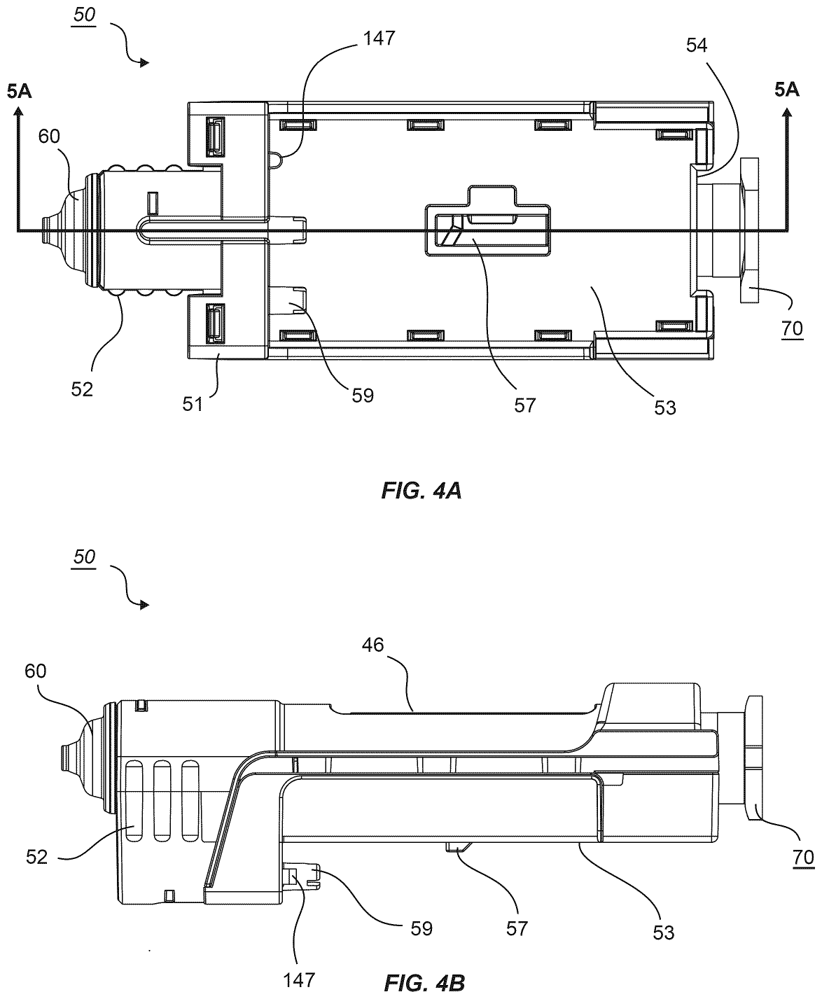

Referring to FIG. 2A, a forward perspective view of cartridge 50 is provided while FIG. 2B depicts a rear perspective view of cartridge 50. Similarly, FIG. 3A depicts a lower rear perspective view of cartridge 50. FIG. 3B depicts a top view of cartridge 50, FIG. 4A depicts a bottom view of cartridge 50, and FIG. 4B depicts a side-plan view of cartridge 50. Cartridge housing 49 can include a lower surface 53, a rear face 54, and a forward stepped portion 51 opposite the rear face 54. The syringe 70 can be slidable through an opening of face 54. The housing 49 can be a multi-part shell with an aligning groove 55 that can engage with cartridge chamber 27. Housing 49 can be formed and/or assembled in a number of ways, including but not limited to, machining, molding, injection molding, three-dimensional printing, or any other suitable manufacturing process.

Suitable materials for housing 49 may include one or more of glass filled nylon, glass filled polypropylene, glass filled polyethylene, polypropylene, polyethylene, or a plastic material. In some examples, housing 49 can include two or more sections of moldable plastic, e.g., a first half and a second half. The sections of housing 49 can be assembled together with fasteners (e.g., screws, rivets, a weld [e.g., a sonic weld], one or more straps or snaps, an adhesive or adhesive tape, etc.) such that the internal components are disposed between the portions and/or respective halves of housing 49. In some aspects, housing 49 can include a housing spray outlet associated with nozzle assembly that enables charged treatment solution to be expelled from cartridge 50. Housing 49 can include a window 46 so as to visualize aspects of syringe 70. Window 46 can be a cutout etched into housing 49 and/or include one or more transparent portions so as to view aspects of syringe 70, such as position of plunger 71, levels of treatment solution, etc. Housing 49 can include one or more tactile nozzle extrusions 52 adjacent the nozzle assembly. The extrusions 52 can be configured to induce gripping from users.

Groove 55 can engage with and align the housing 49 with the cartridge chamber 27. Housing 49 can include one groove 55 positioned on opposite lateral sides of cartridge 50. As specifically shown in FIG. 3A, the cartridge 50 can include a high voltage (HV) contact (e.g., contact 147b of connector 147) and one or more air supply ports 59. HV contact 147 can be configured to flex or otherwise move and electrically connect with a HV contact port positioned on an HV wall of chamber 27. The air supply port 59 can be configured to fluidly connect with a corresponding air supply of the applicator (e.g., an air supply port or tube fluidly connected to the air supply pump within applicator 100). Air supply port 59 may be positioned on the stepped portion 51 of housing 49 which is shaped to correspondingly attach to a stepped portion of the chamber 27.

Once securely engaged with chamber 27, cartridge 50 can be released by a release button 57 shown in FIG. 3A. Button 57 can extend at least partially though lower surface 53 of housing 49 via an aperture in the lower surface 53 of housing 49. Button 57 can be removed from cartridge 50 when cartridge 50 is ready to be attached to chamber 27. This can indicate to a user that the cartridge 50 has not yet been used and that the contents therein (e.g., liquid treatment solution) has not been used. In some examples, the contents stored within syringe 70 can be sterilized, and release button 57 can indicate that the contents of disposable cartridge 50 are sterile and have not been used (e.g., not yet used on a previous patient). In some aspects, button 57 can prevent syringe 70 and any components thereof from advancing contents therein prior to use. In some aspects, button 57 can a include near field communication (NFC) tag with identifying cartridge information and other operational parameters readable therefrom indicate to a user that cartridge 50 has not yet been used and that the contents therein (e.g., liquid treatment solution) has not been used.

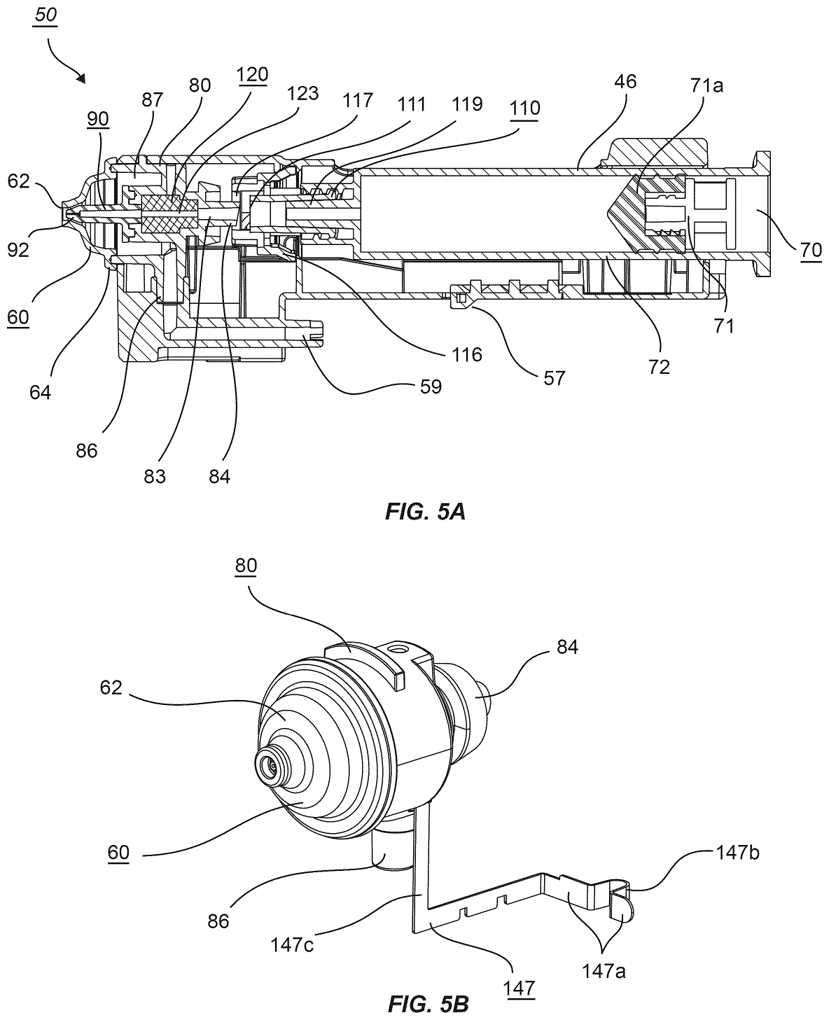

FIG. 5A depicts a cross-sectional side view of section 5A-5A of FIG. 4A. FIG. 5B depicts aspects of assembled view of internal components of cartridge 50 with outer housing 49 removed. Syringe 70 can be an assembly that includes a glass or plastic syringe for storing the treatment solution that is to be applied using the disposable cartridge 50. The contents of syringe 70 can be preloaded (e.g., with a specific treatment solution). Syringe 70 can be assembled within the disposable cartridge 50 pre-filled with the desired treatment solution. At a proximal end, syringe 70 can include the movable plunger 71 and a barrel portion 72 extended distally therefrom. Plunger 71 is configured to advance within barrel portion 72 so as to advance contents of barrel portion 72 through a distal end of portion 72. In operation, syringe 70 can deliver the stored liquid solution at a predetermined rate from barrel portion 72 through the flow pathway distal thereof (e.g., the flow pathway through components of cartridge such as nozzle housing 80, locking mechanism 110, a nozzle tip 90, and a delivery outlet 62 of cap 60 and ultimately emitted by cartridge 50 onto a treatment site). In some aspects, mechanism 110 can connect to the distal end of syringe 70.

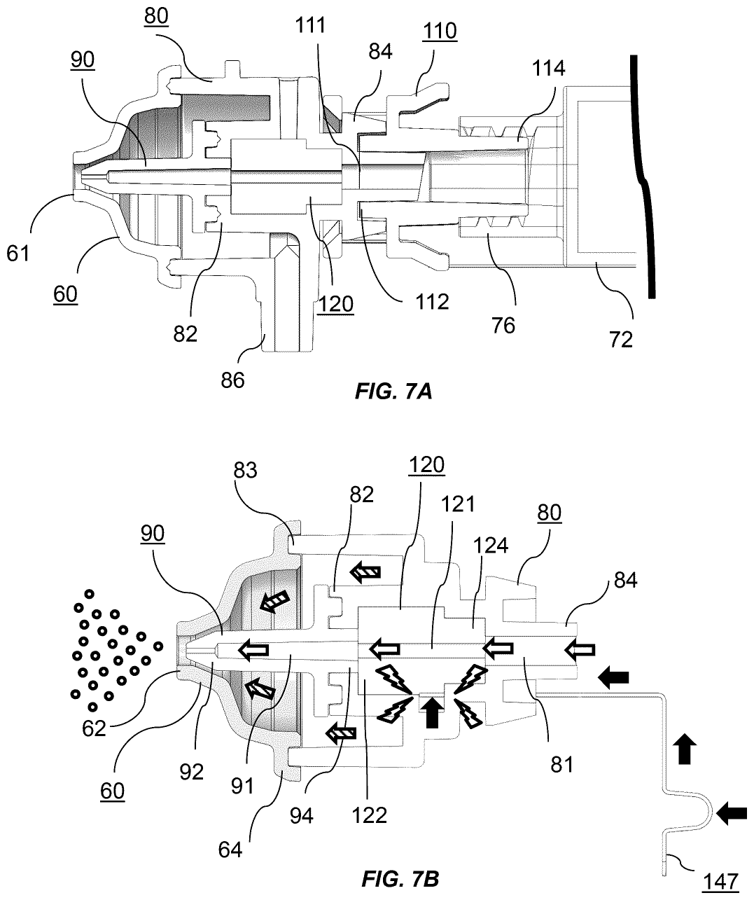

As also shown in FIGS. 5A and 5B, nozzle cap 60 is provided with cartridge 50 and includes a delivery outlet 62 at its distal end. Outlet 62 can include an opening in fluid communication with an opening of an open proximal end 64 that has a larger diameter. Cap 60 can be funnel-shaped, as well as any other shape as needed or required to facilitate egress of contents from syringe 70 through cap 60. Cap 60 can be connected to the nozzle housing 80. Between cap 60 and nozzle housing 80, an atomizing air chamber 87 can be formed. Chamber 87 can include a range of volumes. For example and without limitation, chamber 87 can include a volume of at least approximately 900 mm³. In some examples, chamber 87 can include a volume of at least approximately 100 mm³. In some examples, chamber 87 includes a volume of between at least approximately 100 mm³ and approximately 1,000 mm³. Housing 80 can include a proximal inlet end 84 and an open distal end 82. In some aspects, chamber 87 can be formed by a perimeter of end 64 connecting to a perimeter 82a (see FIG. 7B) of an open distal end 82 of housing 80 (e.g., sealed, hermetically sealed, bonded, welded, adhered, etc.).

As shown most clearly in FIG. 5A, conductive insert 120 can be positioned in housing 80 (e.g., nested therein or otherwise positioned in a cavity of housing 80). In some aspects, insert 120 can be at least partially positioned between ends 82 and 84. Air supply port 59 can extend at least partially from portion 51 of housing 49 and be shaped to fluidly connect with an air supply of applicator 100 (e.g., an air supply pump). Port 59 can also fluidly connect with an air supply port 86 of housing 80. Port 86 can be in fluid communication with chamber 87 for atomizing electrostatically charged treatment solution from cartridge 50 (e.g., fluid egressing from syringe 70 and ultimately through outlet 62 of cap 60). Intake of air at port 86 can provide high velocity air to chamber 87 to spray (e.g., droplets 38 of FIG. 7B) the contents of cartridge 50 (e.g., contents disposed in syringe 70) to the treatment site. Insert 120 can include a fluid channel portion 123 of a flow pathway formed at least partially between cap 60, housing 80, and insert 120. In some aspects, the flow pathway can include the fluid channel 123 and a fluid channel 83 of housing 80 proximal thereof.

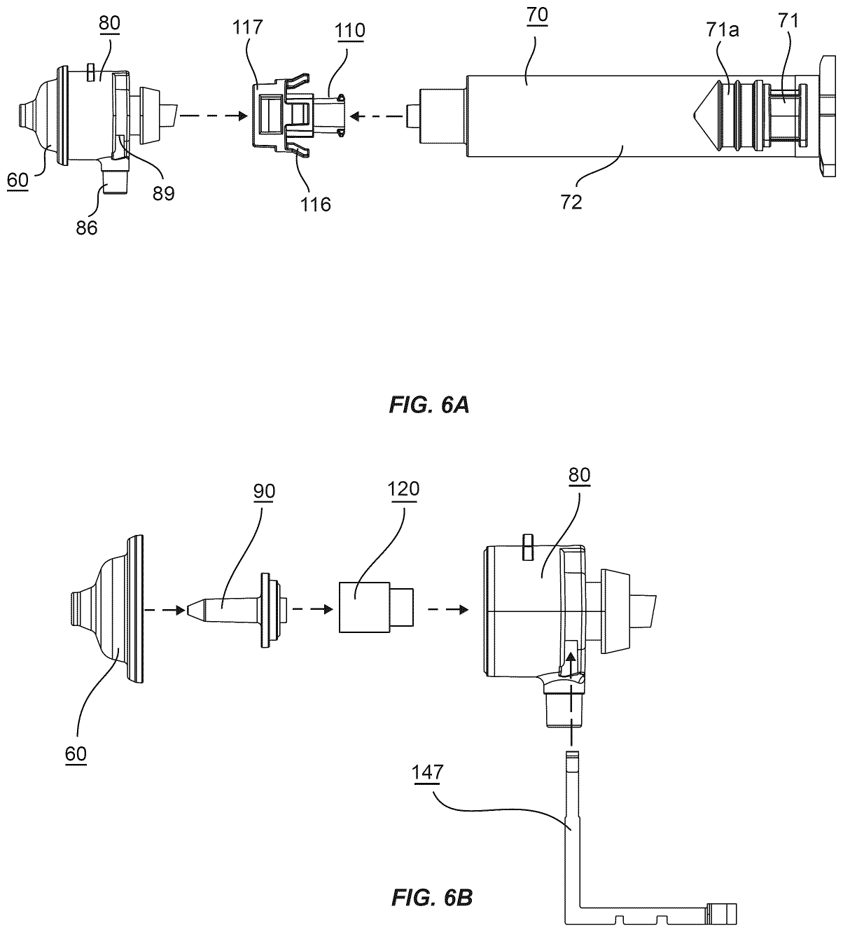

As shown most clearly in FIG. 5B, a high voltage connector 147 can be extended at least partially through housing 80 and electrically connected to insert 120. Connector 147 can be configured to electrically connect with a high voltage contact of applicator 100 (e.g., positioned in chamber 27), that in turn is electrically connected to the high voltage module of applicator 100. By electrically connecting to the high voltage module, connector 147 can electrically connect with insert 120 so as to electrostatically charge fluid contents within channel 123 of insert 120. In some aspects, connector 147 can include a thin conductive spring like material. In some aspects, connector 147 can be spring-like or otherwise biased and/or shaped to extend at least partially through a high voltage port 89 (see, e.g., FIG. 6A) of housing 80. Connector 147 can include a first segment 147a orthogonal or otherwise angled relative to a second segment 147c (e.g., approximately 45 degrees, approximately 30 degrees, approximately 90 degrees, approximately 135 degrees, etc.).

As shown in FIG. 3A extending from an aperture of portion 51 as well as in FIGS. 5B, 6B, and 7B, segment 147a can include an outward flexed contact 147b configured to electrically connect with a high voltage contact of applicator 100 (e.g., a contact in chamber 27). In some aspects, contact 147b can be formed between two opposing ends of segment 147a. Contact 147b can be formed with a resilient or spring-like force that can be exerted in response to present against contact 147b during use. Contact 147b can be substantially curved to create an externally accessible contact surface to mechanically contact and flex inwardly but apply an outward spring-like force to maintain contact with the corresponding high voltage contact of the applicator 100. While only the certain bends and curves of connector 147 are shown herein, connector 147 can include any shape needed or required.

As depicted in the top cross-sectional view of FIG. 7B specifically, connector 147 is shown having been inserted through high voltage port 89 of housing 80 until contacting high voltage contact 143. Contact 143 in turn can physically contact insert 120 so that voltage is applied to fluid passing through pathway 121. Operationally, the uncolored schematic arrows of FIG. 7B denote treatment solution flowing from syringe 70 through the flow pathways of mechanism 110, insert 120, tip 90, and egressing through outlet 62 of cap 60. In some aspects, to atomize charged treatment solution from syringe 70 and ultimately through cap 60, the hatched schematic arrows of FIG. 7B denote air flowing from the air supply of the applicator 100 through air supply port 86 into chamber 87 until outlet 62. The dark schematic arrows of FIG. 7B denote high voltage being applied to contact 147b then traveling along connector 147 to contact 143 to apply the high voltage to the flow pathway of cartridge 50 (e.g., to insert 120).

In some aspects, since unlike charges attract each other, the charging by insert 120 with connector 147 can be positive or it can be negative. In one example, a negative charge can be used with the emission of cartridge 50 (e.g., electrostatically charged contents from syringe 70). In this example, atomized droplets 38 as shown in FIG. 7B sprayed out through outlet 62 can carry a negative charge and positive charge can be induced on the intended object (e.g., via ground to attract the negative charges droplets). The negative charge on the droplets can be neutralized by induced positive charge on the intended object when it hits the surface of the intended object.

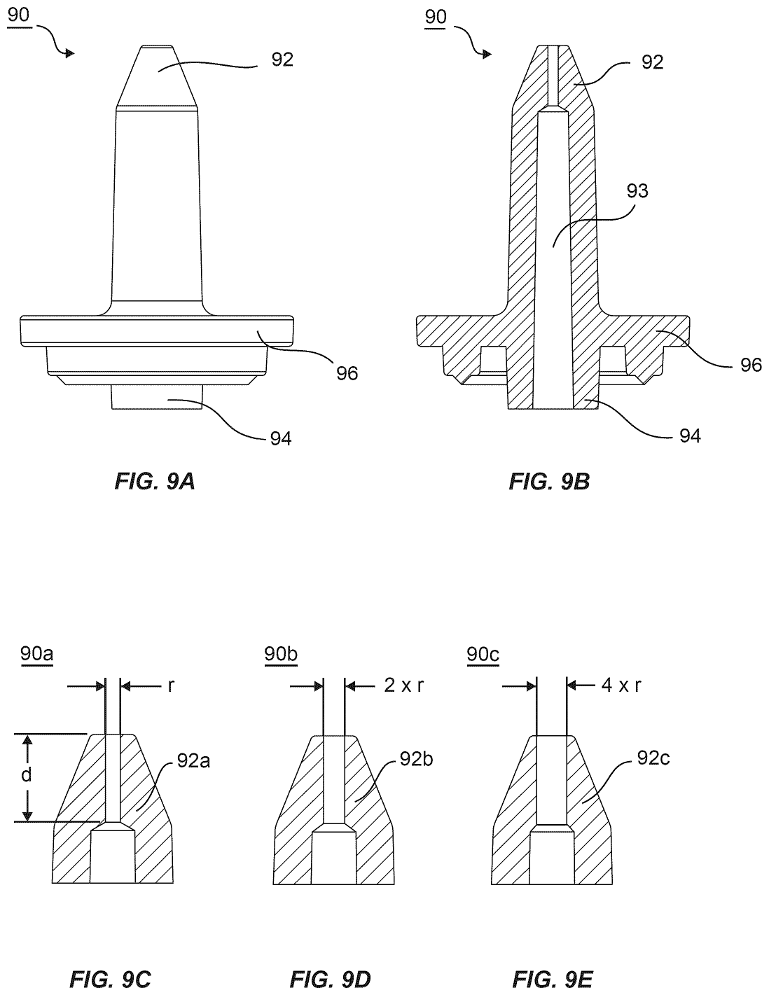

In some aspects as in FIG. 5A and FIG. 6B, a nozzle tip 90 can be positioned between cap 60 and housing 80. As illustrated in both FIGS. 5A, 6B and 9A, tip 90 can include a delivery outlet 92 at a distal end and a proximal inlet 94 opposite outlet 92. A washer surface 96 can be positioned between outlet 92 and inlet 94. In some aspects, surface 96 can be positioned adjacent or otherwise near inlet 94. As shown in FIG. 9B, portion 93 of the collective flow pathway of cartridge 50 can extend at least partially between inlet 94 and outlet 92. Outlet 92 can include a portion with a diameter smaller than pathway 93. In some aspects, pathway 93 can be in fluid communication with pathway 123 of insert 120. During operation, air can be directed through ports 59 and 86 into chamber 87 and is able to flow around tip 90. Air can flow around outlet 92 in the area 81 between an outer surface of outlet 92 and an inner surface of outlet 62 of cap 60 and egress distally therefrom to atomize fluid egressing from pathway 93 through outlet 92.

Additional exemplary nozzle tips 90 are illustrated in FIG. 9C to FIG. 9E. Specifically, FIGS. 9C to 9E depict example outlet portions of examples tips 90a, 90b, 90c with differing outlet diameters. Specifically, the outlet portion 92a of FIG. 9C includes a diameter r whereas FIG. 9D depicts an example outlet portion 92b having a diameter of 2 x r, where r represents an exemplary diameter associated with unit r of tip 90a. Accordingly, diameter of the tip 92b is twice or 2 x r and FIG. 9E depicts an example outlet portion 92c having a diameter of four times or 4 x r. FIGS. 9C to 9E are merely example outlet tube sizes and any size or shape contemplated as needed or required. In some aspects, a diameter of outlet portion can be specific to the fluid being sprayed. In some aspects, nozzle tip diameters can range from approximately 16-gauge to approximately 25-gauge tube sizes.

Turning now to FIGS. 6A and 6B, certain internal components of cartridge 50 are depicted in an exploded state. Specifically, FIG. 6A shows the nozzle cap 60 of cartridge 50 connected to nozzle housing 80 prior to being connected with locking mechanism 110. Locking mechanism 110 is shown prior to being connected to syringe 70. In some aspects, a distal delivery outlet of syringe 70 can be inserted into an inner portion of the flow pathway of mechanism 110 so that threads 76 of syringe 70 and mechanism 110 can connect with each other. FIG. 6B similarly shows nozzle tip 90 prior to its distal end being inserted into an inner portion of cap 60 and the proximal end of tip 90 prior to being inserted and/or otherwise connected so as to press against conductive insert 120. FIG. 6B also shows insert 120 just prior to being at least partially positioned within housing 80 and connector 147 just prior to being assembled with housing 80 so as to electrically connect with insert 120 once insert 120 is at least partially positioned within housing 80.

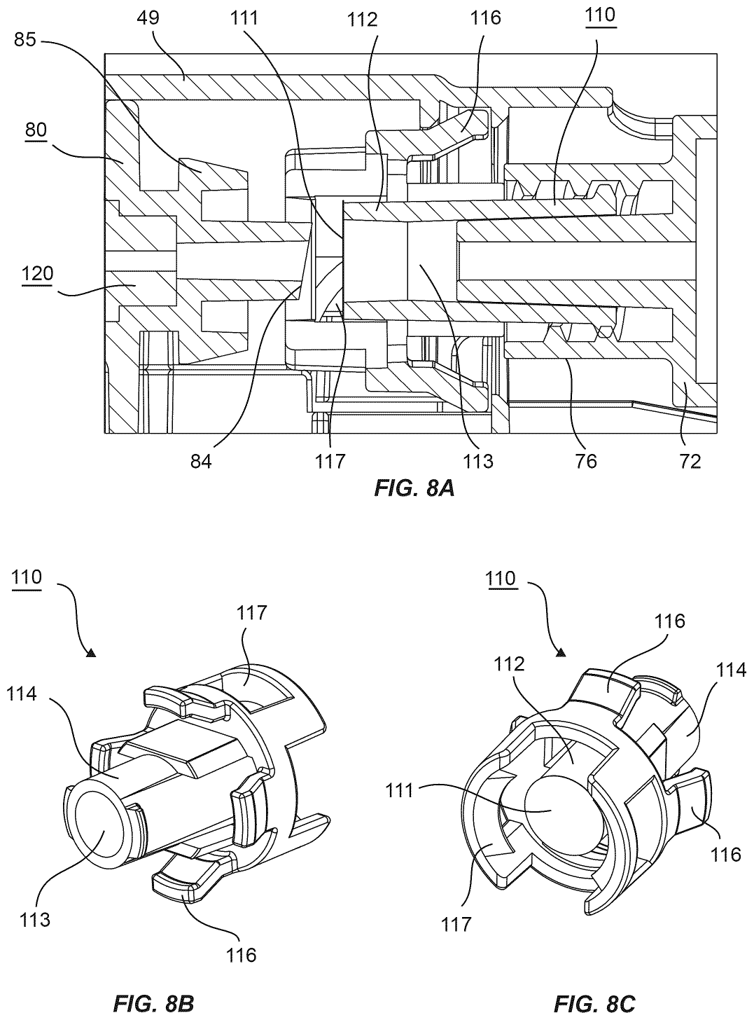

Mechanism 110 can be positioned between the distal end 74 of syringe 70 and end 84 of the nozzle housing 80. In some aspects, proximal end 114 of mechanism 110 can only an outer threaded surface configured to connect with inner threads 76 of syringe 70. In some aspects, end 114 can include one or more luer locking tabs, as shown in FIG. 8B. In some aspects, mechanism 110 can include at least one retainer tab 116 to securely connect with the distal end of syringe 70 and at least one snap connector 117 to releasably snap onto the nozzle housing 80. Tab 116 can be configured prevent syringe 70 from removed from the disposable fluid delivery system In some aspects, connector 117 can be configured to flex over and then snap onto an outer angled guide 85 of housing 80 adjacent end 84. In the example of FIG. 5A, mechanism 110 is shown in a stored position just prior to being urged distally whereby connector 117 while radially flex outward as it slides over guide 85 until snapping into place when mechanism 110 is secured with housing 80.

FIG. 7A depicts a close-up, side cross-section view showing aspects of the cartridge 50 in an assembled state while FIG. 7B depicts a close-up, side cross-section view showing aspects of the example cartridge of FIGS. 1A to 2B in an assembled, operational state. In the example of FIG. 7A, mechanism 110 is shown in an operational ready position since mechanism 110 has been urged distally so that connector 117 is now distal of guide 85 so that catch surface 112 of mechanism 110 is pressed against an inner receiver adjacent guide 85. Aspects of mechanism 110 are more particularly shown in FIGS. 8A to 8C. In particular, FIG. 8A depicts a close-up, side cross-section view showing mechanism 110 assembled between the end 76 of syringe 70 and proximal end 84 of housing 80. FIG. 8B depicts a forward perspective view of mechanism 110 while FIG. 8C depicts a rear perspective view of mechanism 110.

As shown in FIGS. 8B to 8C, mechanism 110 can one or more retainer tabs 115 configured to securely connect with end 76 of syringe 70. In the arrangement shown, mechanism 110 includes four tabs 115 though any number of tabs are contemplated, including as few as one tab 115 and more than four tabs 115. Mechanism 110 can also include at least one snap connector 117 configured to slide over guide 85 and then snap into a space proximal of guide 85. Mechanism 110 can also include a flow pathway 113 that forms part of the flow pathway of cartridge. In the operation of urging mechanism 110 distally (e.g., by syringe 70 moving mechanism 110 distally), a corresponding foil seal 111 can be pierced by end 84 to permit egress of contents of syringe 70 into housing 80. Seal 111 in some aspects is an openable seal that seals an inner hollow portion 113 of mechanism 110. In this respect, portion 113 forms a portion of the flow pathway defined between corresponding hollowed flow portions (e.g., portion 83 of housing 80, portion 123 of insert 120, and portion 93 of tip 90). In some aspects, portions 113, 83, 123, and 93 are in fluid communication with each other to form the flow pathway between syringe 70 and delivery outlet 62.

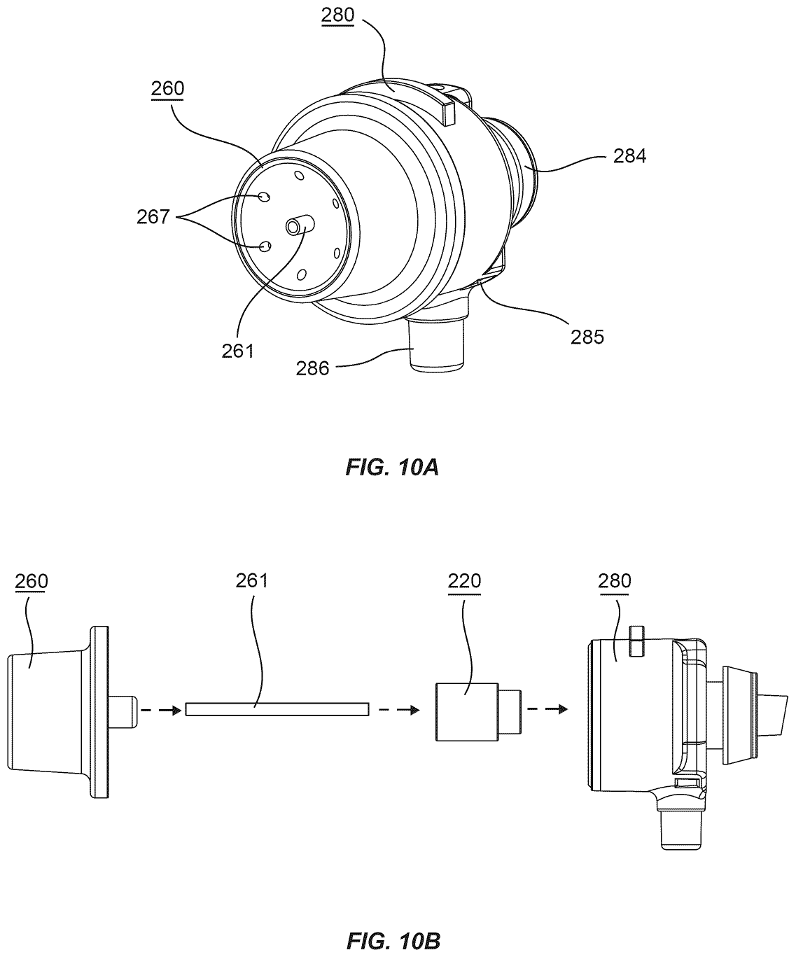

Referring to FIG. 10A, a forward perspective view of internal components that form aspects of the nozzle system of another exemplary cartridge for electrospinning is shown. In FIG. 10A the assembled internal components shown are without the cartridge outer housing for illustrative purposes only though are assembled within a similar cartridge housing, such as housing 49, during operation. FIG. 10B depicts a side exploded view of the assembled components of FIG. 10A. As shown, the example electrospinning cartridge of FIGS. 10A and 10B can include a nozzle cap 260 that connects to an inner delivery tube 261, conductive insert 120, and nozzle housing 280. Aspects of cartridge of the components shown in FIGS. 10A and 10B include like numerals as in previously discussed cartridge 50 indicate like structural elements and features.

In some aspects, nozzle cap 260 of FIG. 10A is configured to receive source liquid from syringe 270 and feed high-compressed air via one or more injection holes 267 so that the source liquid of syringe 270 for fiber can be injected together with air and a voltage applied to at least a portion of the flow pathway to produce and emit electrospun fibers having fine diameters. Similar to syringe 70, syringe 270 can be an assembly that includes a glass or plastic syringe for storing the liquid solution that is to be applied using the disposable cartridge 250. Syringe 270 can be assembled within the disposable cartridge pre-filled with the desired liquid solution. In operation, syringe 270 can be configured to deliver the electrospun fiber from the stored liquid solution at a predetermined rate from barrel portion 272 through delivery tube 261 and ultimately emitted onto a treatment site (e.g., a wound site of a patient). Tube 261 can be a tubular insert configured to be positioned through the flow pathway of cap 260 and aspects of housing 280 and insert 220. In some aspects, tube 261 can range from approximately 16-gauge to approximately 25-gauge tube sizes.

Cap 260 can include a nozzle outlet channel through which tube 261 can pass and around which holes 267 can be radially positioned. As shown in FIG. 10A, housing 260 can include six (6) holes radially arranged around a common axis of the outlet channel and/or tube 261, though fewer or greater number of holes can be used as needed or required. In some aspects, holes 267 can be replaced by an outer concentric outlet channel that completely surrounds tube 261 and is similarly in fluid communication with port 266. During operation, air pumped through port 266 into chamber 287 and out through holes 267 can create an air curtain around the electrospun fiber being emitted from tube 261. In this respect, air curtain prevents outside air from affecting the emitted fiber during the electrospinning.

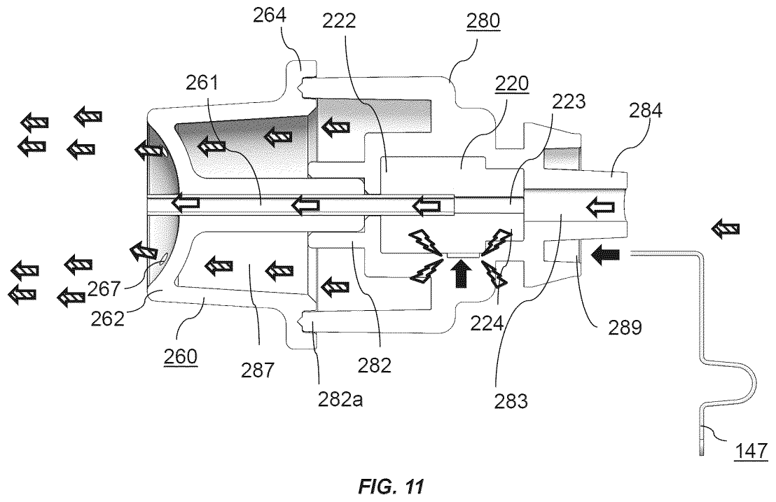

In the depicted example of FIGS. 10A, 10B, and 11, holes 267 are shaped substantially straight or otherwise angled in parallel and/or axially aligned with tube 261 so that the shape of the corresponding air curtain is parallel and/or axially aligned. In other examples, holes 267 are shaped angled upwards relative to a longitudinal axis of tube 261 so as to create a funnel shaped, tapered outward air curtain. In other examples, holes 267 are shaped angled downwards relative to a longitudinal axis of tube 261 so as to create a converging, tapered inward shaped air curtain. In some aspects, holes 267 can be radially arranged around tube 261 and formed in cap 260. FIG. 11 is a top cross-section view of the assembled components of FIG. 10A. Specifically, FIG. 11 shows cap 260 connecting with housing 280 about respective perimeters 263, 283 to form the internal atomizing chamber 287. Just as in port 86 of cartridge 50, port 286 provides intake of air into chamber 287 from the internal air supply of applicator 100 and connector 147 can electrically connect with insert 220.

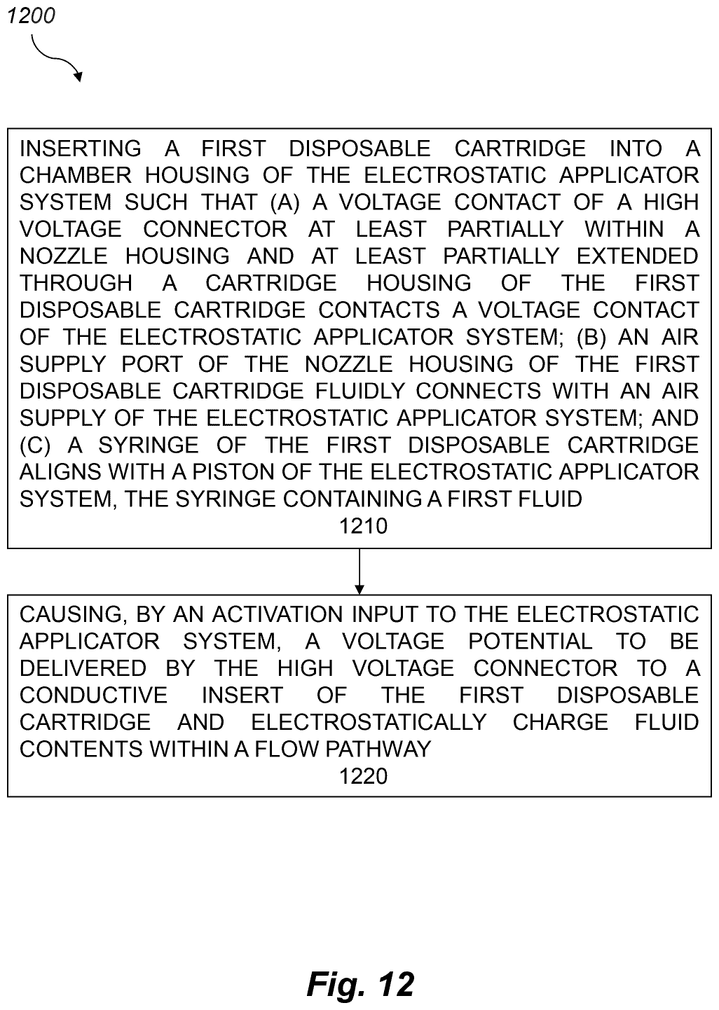

FIG. 12 is a method 1200 for operating an electrostatic applicator system. Step 1210 of method 1200 can include inserting a first disposable cartridge into a chamber housing of the electrostatic applicator system such that (a) a voltage contact of a high voltage connector at least partially within a nozzle housing and at least partially extended through a cartridge housing of the first disposable cartridge contacts a voltage contact of the electrostatic applicator system; (b) an air supply port of the nozzle housing of the first disposable cartridge fluidly connects with an air supply of the electrostatic applicator system; and (c) a syringe of the first disposable cartridge aligns with a piston of the electrostatic applicator system. Step 1220 of method 1200 can include causing, by an activation input to the electrostatic applicator system, a voltage potential to be delivered by the high voltage connector to a conductive insert of the first disposable cartridge and electrostatically charge fluid contents within a flow pathway.

It is also to be understood that the mention of one or more steps of method 1200 does not preclude the presence of additional method steps or intervening method steps between those steps expressly identified. Steps of method 1200 may be performed in a different order than those described herein without departing from the scope of the disclosed technology. For example, method 1200 can include forming an atomizing air chamber between a nozzle cap and a nozzle housing of the first disposable cartridge to atomize electrostatically charged fluid contents from the flow pathway and egressing through a delivery outlet of the nozzle cap. Method 1200 can also include directing air, from a pump of the electrostatic applicator system, through an air supply port of the nozzle housing and into the atomizing air chamber; and causing, by moving the plunger, the electrostatically charged fluid contents from the syringe to delivered through a delivery outlet of the nozzle cap.

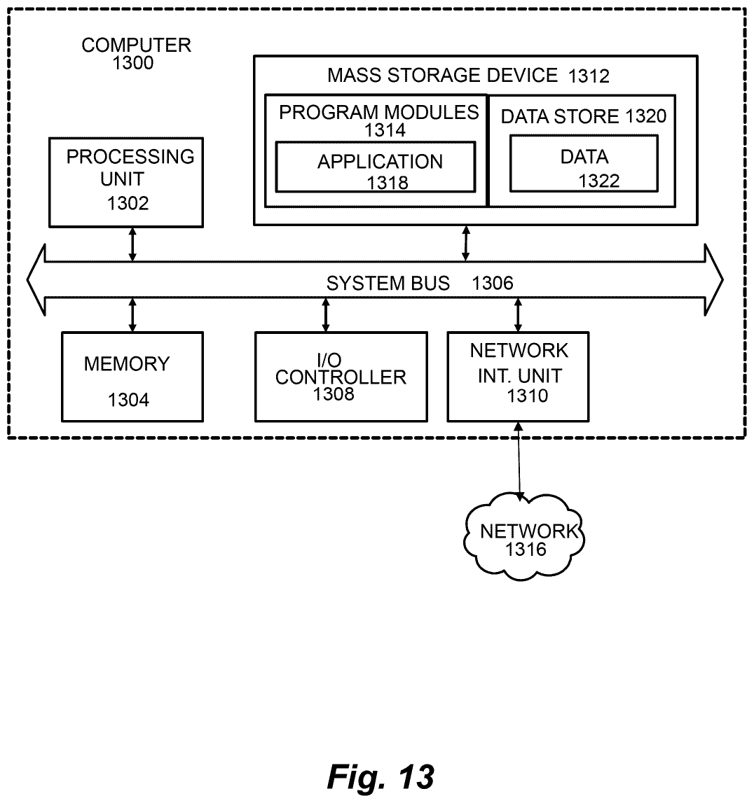

FIG. 13 is a computer architecture diagram showing a general computing system capable of implementing aspects of the present disclosure in accordance with one or more embodiments described herein, such as the disposable cartridges and electrostatic applicators described herein. In any of these example implementations, computer 1300 of the aforementioned may be configured to perform one or more functions associated with embodiments of this disclosure. For example, the computer 1300 may be configured to perform operations in accordance with those examples shown in FIGS. 1A to 12. It should be appreciated that the computer 1300 may be implemented within a single computing device or a computing system formed with multiple connected computing devices. The computer 1300 may be configured to perform various distributed computing tasks, in which processing and/or storage resources may be distributed among the multiple devices. The data acquisition and display computer 1350 and/or operator console 1310 of the system shown in FIG. 13 may include one or more systems and components of the computer 1300.

As shown, the computer 1300 includes a processing unit 1302 (“CPU”), a system memory 1304, and a system bus 1306 that couples the memory 1304 to the CPU 1302. The computer 1300 further includes a mass storage device 1312 for storing program modules 1314. The program modules 1314 may be operable to analyze data from any herein disclosed components (e.g., whether a cartridge is assembled properly, information related to contents of a syringe, operational parameters such as flow rate, applied voltage, user information, patient information, operational status, battery levels, flow pattern, etc.) and/or control any related operations (e.g., transmitting one or more messages to users, etc.). The program modules 1314 may include an application 1318 for performing data acquisition and/or processing functions as described herein, for example to acquire and/or process any of the herein discussed data feeds. The computer 1300 can include a data store 1320 for storing data that may include data 1322 of data feeds from system components.

The mass storage device 1312 is connected to the CPU 1302 through a mass storage controller (not shown) connected to the bus 1306. The mass storage device 1312 and its associated computer-storage media provide non-volatile storage for the computer 1300. Although the description of computer-storage media contained herein refers to a mass storage device, such as a hard disk or CD-ROM drive, it should be appreciated by those skilled in the art that computer-storage media can be any available computer storage media that can be accessed by the computer 1300.

By way of example and not limitation, computer storage media (also referred to herein as “computer-readable storage medium” or “computer-readable storage media”) may include volatile and non-volatile, removable and non-removable media implemented in any method or technology for storage of information such as computer-storage instructions, data structures, program modules, or other data. For example, computer storage media includes, but is not limited to, RAM, ROM, EPROM, EEPROM, flash memory or other solid state memory technology, CD-ROM, digital versatile disks (“DVD”), HD-DVD, BLU-RAY, or other optical storage, magnetic cassettes, magnetic tape, magnetic disk storage or other magnetic storage devices, or any other medium which can be used to store the desired information and which can be accessed by the computer 1300. “Computer storage media”, “computer-readable storage medium” or “computer-readable storage media” as described herein do not include transitory signals.

According to various embodiments, the computer 1300 may operate in a networked environment using connections to other local or remote computers through a network 1316 via a network interface unit 1310 connected to the bus 1306. The network interface unit 1310 may facilitate connection of the computing device inputs and outputs to one or more suitable networks and/or connections such as a local area network (LAN), a wide area network (WAN), the Internet, a cellular network, a radio frequency (RF) network, a Bluetooth-enabled network, a Wi-Fi enabled network, a satellite-based network, or other wired and/or wireless networks for communication with external devices and/or systems.

The computer 1300 may also include an input/output controller 1308 for receiving and processing input from any of a number of input devices. Input devices may include one or more of keyboards, mice, stylus, touchscreens, microphones, audio capturing devices, and image/video capturing devices. An end user may utilize the input devices to interact with a user interface, for example a graphical user interface, for managing various functions performed by the computer 1300. The bus 1306 may enable the processing unit 1302 to read code and/or data to/from the mass storage device 1312 or other computer-storage media.

The computer-storage media may represent apparatus in the form of storage elements that are implemented using any suitable technology, including but not limited to semiconductors, magnetic materials, optics, or the like. The computer-storage media may represent memory components, whether characterized as RAM, ROM, flash, or other types of technology. The computer storage media may also represent secondary storage, whether implemented as hard drives or otherwise. Hard drive implementations may be characterized as solid state or may include rotating media storing magnetically-encoded information. The program modules 1314, which include the data feed application 1318, may include instructions that, when loaded into the processing unit 1302 and executed, cause the computer 1300 to provide functions associated with one or more embodiments illustrated in the figures of this disclosure. The program modules 1314 may also provide various tools or techniques by which the computer 1300 may participate within the overall systems or operating environments using the components, flows, and data structures discussed throughout this description.

In general, the program modules 1314 may, when loaded into the processing unit 1302 and executed, transform the processing unit 1302 and the overall computer 1300 from a general-purpose computing system into a special-purpose computing system. The processing unit 1302 may be constructed from any number of transistors or other discrete circuit elements, which may individually or collectively assume any number of states. More specifically, the processing unit 1302 may operate as a finite-state machine, in response to executable instructions contained within the program modules 1314. These computer-executable instructions may transform the processing unit 1302 by specifying how the processing unit 1302 transitions between states, thereby transforming the transistors or other discrete hardware elements constituting the processing unit 1302.

Encoding the program modules 1314 may also transform the physical structure of the computer-storage media. The specific transformation of physical structure may depend on various factors, in different implementations of this description. Examples of such factors may include but are not limited to the technology used to implement the computer-storage media, whether the computer storage media are characterized as primary or secondary storage, and the like. For example, if the computer storage media are implemented as semiconductor-based memory, the program modules 1314 may transform the physical state of the semiconductor memory, when the software is encoded therein. For example, the program modules 1314 may transform the state of transistors, capacitors, or other discrete circuit elements constituting the semiconductor memory.

As another example, the computer storage media may be implemented using magnetic or optical technology. In such implementations, the program modules 1314 may transform the physical state of magnetic or optical media, when the software is encoded therein. These transformations may include altering the magnetic characteristics of particular locations within given magnetic media. These transformations may also include altering the physical features or characteristics of particular locations within given optical media, to change the optical characteristics of those locations. Other transformations of physical media are possible without departing from the scope of the present description, with the foregoing examples provided only to facilitate this discussion.

According to certain embodiments, the above-described data feeds may be stored in databases such as database servers that store master data, event related data, response plan data, telemetry information, and mission data as well as logging and trace information. The databases may also provide an API and/or API access (e.g., for open source) to the web server for data interchange based on JSON specifications. In some embodiments, the database may also directly interact with systems and monitoring devices to identify, determine, and control response operations. According to certain embodiments, the database servers may be optimally designed for storing large amounts of data, responding quickly to incoming requests, having a high availability and historizing master data.

In the description herein, numerous specific details are set forth. However, it is to be understood that embodiments of the present disclosure may be practiced without these specific details. In other instances, well-known methods, structures, and techniques have not been shown in detail in order not to obscure an understanding of this description. References to “one embodiment,” “an embodiment,” “example embodiment,” “some embodiments,” “certain embodiments,” “various embodiments,” etc., indicate that the embodiment(s) of the present disclosure so described may include a particular feature, structure, or characteristic, but not every embodiment necessarily includes the particular feature, structure, or characteristic. Further, repeated use of the phrase “in one embodiment” does not necessarily refer to the same embodiment, although it may.

Throughout the specification and the claims, the following terms take at least the meanings explicitly associated herein, unless the context clearly dictates otherwise. The term “or” is intended to mean an inclusive “or.” Further, the terms “a,” “an,” and “the” are intended to mean one or more unless specified otherwise or clear from the context to be directed to a singular form. Accordingly, “a nozzle housing” or “the nozzle housing” may refer to one or more nozzle housings where applicable.

Unless otherwise specified, the use of the ordinal adjectives “first,” “second,” “third,” etc., to describe a common object, merely indicate that different instances of like objects are being referred to, and are not intended to imply that the objects so described must be in a given sequence, either temporally, spatially, in ranking, or in any other manner.

Certain embodiments of the present disclosure are described above with reference to block and flow diagrams of systems and methods and/or computer program products according to example embodiments of the present disclosure. It will be understood that one or more blocks of the block diagrams and flow diagrams, and combinations of blocks in the block diagrams and flow diagrams, respectively, may be implemented by computer-executable program instructions. Likewise, some blocks of the block diagrams and flow diagrams may not necessarily need to be performed in the order presented, or may not necessarily need to be performed at all, according to some embodiments of the present disclosure.

These computer-executable program instructions may be loaded onto a general-purpose computer, a special-purpose computer, a processor (e.g., a processor chip, single/multi-processor architectures, sequential (Von Neumann)/parallel architectures, and specialized circuits, etc.), or other programmable data processing apparatus to produce a particular machine, such that the instructions that execute on the computer, processor, or other programmable data processing apparatus create means for implementing one or more functions specified in the flow diagram block or blocks. These computer program instructions may also be stored in a computer-readable memory that may direct a computer or other programmable data processing apparatus to function in a particular manner, such that the instructions stored in the computer-readable memory produce an article of manufacture including instruction means that implement one or more functions specified in the flow diagram block or blocks.

As an example, embodiments of the present disclosure may provide for a computer program product, including a computer-usable medium having a computer-readable program code or program instructions embodied therein, said computer-readable program code adapted to be executed to implement one or more functions specified in the flow diagram block or blocks. The computer program instructions may also be loaded onto a computer or other programmable data processing apparatus to cause a series of operational elements or steps to be performed on the computer or other programmable apparatus to produce a computer-implemented process such that the instructions that execute on the computer or other programmable apparatus provide elements or steps for implementing the functions specified in the flow diagram block or blocks.

Accordingly, blocks of the block diagrams and flow diagrams support combinations of means for performing the specified functions, combinations of elements or steps for performing the specified functions and program instruction means for performing the specified functions. It will also be understood that each block of the block diagrams and flow diagrams, and combinations of blocks in the block diagrams and flow diagrams, may be implemented by special-purpose, hardware-based computer systems that perform the specified functions, elements or steps, or combinations of special-purpose hardware and computer instructions.

Various aspects described herein may be implemented using standard programming and/or engineering techniques to produce software, firmware, hardware, and/or any combination thereof to control a computing device to implement the disclosed subject matter. A computer-readable medium may include, for example: a magnetic storage device such as a hard disk, a floppy disk or a magnetic strip; an optical storage device such as a compact disk (CD) or digital versatile disk (DVD); a smart card; and a flash memory device such as a card, stick or key drive, or embedded component. Additionally, it should be appreciated that a carrier wave may be employed to carry computer-readable electronic data including those used in transmitting and receiving electronic data such as streaming video or in accessing a computer network such as the Internet or a local area network (LAN). Of course, a person of ordinary skill in the art will recognize many modifications may be made to this configuration without departing from the scope or spirit of the claimed subject matter.

While certain embodiments of the present disclosure have been described in connection with what is presently considered to be the most practical and various embodiments, it is to be understood that the present disclosure is not to be limited to the disclosed embodiments, but on the contrary, is intended to cover various modifications and equivalent arrangements included within the scope of the appended claims. Although specific terms are employed herein, they are used in a generic and descriptive sense only and not for purposes of limitation.

This written description uses examples to disclose certain embodiments of the present disclosure, including the best mode, and also to enable any person skilled in the art to practice certain embodiments of the present disclosure, including making and using any devices or systems and performing any incorporated methods. The patentable scope of certain embodiments of the present disclosure is defined in the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

The specific configurations, choice of materials and the size and shape of various elements can be varied according to particular design specifications or constraints requiring a system or method constructed according to the principles of the disclosed technology. Such changes are intended to be embraced within the scope of the disclosed technology. The presently disclosed embodiments, therefore, are considered in all respects to be illustrative and not restrictive. It will therefore be apparent from the foregoing that while particular forms of the disclosure have been illustrated and described, various modifications can be made without departing from the spirit and scope of the disclosure and all changes that come within the meaning and range of equivalents thereof are intended to be embraced therein.

The following clauses list non-limiting embodiments of the disclosure:

1. A disposable fluid delivery system for an electrostatic applicator, including:
a nozzle cap including a distal end including a delivery outlet and an open proximal end;
a nozzle housing including a proximal inlet end and an open distal end connected to the open proximal end of the nozzle cap;
a conductive insert at least partially positioned between the open distal end and the proximal inlet end and including a portion of a flow pathway formed at least partially between the nozzle cap, the nozzle housing, and the conductive insert; and
a high voltage connector extended at least partially through the nozzle housing and electrically connected to the conductive insert and configured to be in electrical communication with a high voltage module and electrostatically charge fluid contents within the flow pathway.

2. The disposable fluid delivery system of Clause 1, wherein the high voltage connector includes a first segment orthogonal relative to a second segment, wherein at least one of the first and second segments includes an outward flexed contact configured to electrically connect with a high voltage contact of the electrostatic applicator.

3. The disposable fluid delivery system of Clause 1, wherein the high voltage connector includes a first segment angled relative to a second segment, wherein at least one of the first and second segments includes an outward flexed contact configured to electrically connect with a high voltage contact of the electrostatic applicator.

4. The disposable fluid delivery system of Clause 1, wherein the high voltage connector is a spring.

5. The disposable fluid delivery system of Clause 1, wherein the nozzle housing is hermetically sealed to the nozzle cap forming an atomizing air chamber.

6. The disposable fluid delivery system of Clause 5, further including:
a nozzle tip positioned between the nozzle cap and the nozzle housing, the nozzle tip including a distal end aligned with the delivery outlet and a proximal end aligned and in fluid communication with the flow pathway of a distal end of the conductive insert,
wherein air from the atomizing air chamber is able to flow around the nozzle tip and egress out of the delivery outlet to atomize fluid egressing from the flow pathway.

7. The disposable fluid delivery system of Clause 5, wherein the atomizing air chamber includes a volume of at least approximately 900 mm³.

8. The disposable fluid delivery system of Clause 5, wherein the nozzle housing includes an air supply port in fluid communication with the atomizing air chamber for atomizing electrostatically charged fluid from the nozzle cap.

9. The disposable fluid delivery system of Clause 5, wherein the nozzle cap includes: a plurality of holes on a face of the distal end and selectively arranged around the delivery outlet, the plurality of holes in fluid communication with the atomizing air chamber.

10. The disposable fluid delivery system of Clause 9, further including:
a delivery tube at least partially extended from the delivery outlet and in fluid communication with the flow pathway.

11. The disposable fluid delivery system of Clause 9, wherein the face of the distal end is curved inwardly or curved outwardly.

12. The disposable fluid delivery system of Clause 9, the nozzle cap is configured so that during use, air pumped from the atomizing air chamber through and out through the plurality of holes creates an air curtain around the electrostatically charged fluid contents egressing from the delivery outlet.

13. The disposable fluid delivery system of Clause 1, wherein the conductive insert is nested in a cavity of the nozzle housing positioned between the open distal end and the proximal inlet.

14. The disposable fluid delivery system of Clause 1, further including: a syringe removably connected to the proximal inlet end of the nozzle housing, the syringe including a barrel portion and a plunger configured to advance fluids from within the barrel portion and through the flow pathway; and
a cartridge housing at least partially enclosing the nozzle housing, the nozzle cap, the high voltage connector, and the syringe.

15. The disposable fluid delivery system of Clause 14, wherein the high voltage connector is a spring extended at least partially through a high voltage port of the nozzle housing and includes a high voltage contact segment extended at least partially through an aperture of the cartridge housing.

16. The disposable fluid delivery system of Clause 14, wherein a contact of the high voltage connector to provide a voltage potential of approximately 1 V to approximately 40 kV to the conductive insert.

17. The disposable fluid delivery system Clause 14, wherein a contact of the high voltage connector is in physical contact with the conductive insert to provide a voltage potential of approximately 1 V to approximately 40 kV to fluid in the flow pathway.

18. The disposable fluid delivery system of Clause 14, wherein the cartridge housing is a moldable plastic material, wherein the cartridge housing includes a plurality of moldable plastic connectable sections.

19. The disposable fluid delivery system of Clause 14, wherein the syringe contains contents including one or more of an antiseptic, a disinfectant solution, an analgesic, an exosome, a biologic, and/or a liquid bandage solution.

20. The disposable fluid delivery system of Clause 14, further including: a locking mechanism between a distal end of the syringe and the proximal inlet end of the nozzle housing, the locking mechanism including at least one retainer tab configured to securely connect with the distal end of the syringe and at least one snap connector configured to releasably snap onto the nozzle housing, wherein the locking mechanism comprises a portion of the flow pathway.

21. The disposable fluid delivery system of Clause 20, wherein the portion of the flow pathway of the locking mechanism includes an openable seal.

22. The disposable fluid delivery system of Clause 20, wherein the at least one retainer tab is configured prevent the syringe from being removed from the disposable fluid delivery system.

23. The disposable fluid delivery system of Clause 14, wherein an outlet diameter of the delivery outlet ranges between approximately a 16-gauge to 25-gauge tube size.

24. The disposable fluid delivery system of Clause 14, further including:
a reusable electrostatic applicator including a cartridge chamber sized and shaped to accept the cartridge housing, the reusable electrostatic applicator including:
a high voltage module configured to be in electrical communication with an at least partially moveable voltage contact of the high voltage connector; and
a piston positioned proximate the cartridge chamber and configured to advance the plunger enclosed in the cartridge housing when the cartridge housing is assembled with the cartridge chamber.

25. The disposable fluid delivery system of Clause 24, wherein the reusable electrostatic applicator includes:
a motor configured to move the piston;
one or more processor that include at least one processing chip; and
memory storing instructions that, when executed by the one or more processors, causes the reusable electrostatic applicator to:
receive an activation signal;
output a control signal to the motor to actuate the piston; and
output a control signal to a switch to provide voltage from the high voltage module to the high voltage connector.

26. The disposable fluid delivery system of Clause 24, wherein the reusable electrostatic applicator includes:
a housing base including a voltage source;
a device housing including the cartridge chamber; and
a handle extending between the housing base and the device housing.

27. An electrostatic applicator system for delivering a treatment solution to a target site, including:
a portable reusable electrostatic applicator including:
a device housing configured to be handheld;
a motor in the device housing configured to drive a piston;
a voltage source in the device housing;
a high voltage module electrically connected to the voltage source; and
a cartridge chamber; and
a disposable cartridge removably insertable in the cartridge chamber, the disposable cartridge including:
a nozzle cap including a distal end including a delivery outlet and a proximal end;
a nozzle housing including a proximal inlet end and an open distal end connected with the open proximal end of the nozzle cap;
a conductive insert between the open distal end and the proximal inlet end and including a portion of a flow pathway formed at least partially between the nozzle cap, the nozzle housing, and the conductive insert;
a high voltage connector extended at least partially through the nozzle housing and electrically connected to the conductive insert and in electrical communication with the high voltage module;
a syringe including a barrel portion and a plunger configured to advance fluids from within the barrel portion and through the flow pathway; and
a cartridge housing at least partially enclosing the nozzle cap, the nozzle housing, the high voltage connector, and the syringe.

28. The electrostatic applicator system of Clause 27, wherein the cartridge chamber includes a wall including a high voltage contact in electrical communication with the high voltage module and an air supply port in fluid communication with a pump positioned in the device housing;
wherein the high voltage connector is in electrical communication with the high voltage contact of the wall; and
wherein the contact of the voltage wire is in physical contact with an outer surface of the conductive insert to provide a voltage potential of approximately 1 V to approximately 40 kV.

29. The electrostatic applicator system of Clause 27, wherein the reusable electrostatic applicator includes:
one or more processors that include at least one processing chip; and
memory storing instructions that, when executed by the one or more processors, causes the reusable electrostatic applicator to:
receive an activation signal; and
output a control signal to a motor controlling (a) a voltage potential from the high voltage module to the conductive insert via the high voltage connector, (b) a position of the piston and the plunger of the disposable cartridge, and/or (c) a pump regulating air flow from the device housing into the air supply port.

30. The electrostatic applicator system of Clause 29, wherein the disposable cartridge includes an integrated memory including information related to one or more operational parameters of contents stored within the syringe and/or another fluid reservoir of the disposable cartridge, wherein the one or more processors of the reusable electrostatic applicator are configured to communicate with the integrated memory to retrieve information related to the contents of the disposable cartridge and control at least one of a flow rate, a voltage potential, and a nozzle setting.

31. The electrostatic applicator system of Clause 30, further including an accelerometer configured to output a movement signal to the one or more processors in response to detecting movement of the electrostatic applicator system; and a display screen on the electrostatic applicator system activated by a wake signal from the one or more processors in response to the one or more processors receiving the movement signal; and wherein the activation signal is a user input into the display screen.

32. The electrostatic applicator system of Clause 27, wherein the high voltage connector includes a first segment orthogonal relative to a second segment, wherein at least one of the first and second segments includes an outward flexed contact configured to electrically connect with a high voltage contact of the electrostatic applicator.

33. The electrostatic applicator system of Clause 27, wherein the high voltage connector includes a first segment angled relative to a second segment, wherein at least one of the first and second segments includes an outward flexed contact configured to electrically connect with a high voltage contact of the electrostatic applicator.

34. The electrostatic applicator system of Clause 27, wherein the high voltage connector is a spring.

35. The electrostatic applicator system of Clause 27, wherein an atomizing air chamber is formed between the nozzle cap and the nozzle housing.

36. The electrostatic applicator system of Clause 35, further including:
a nozzle tip positioned between the nozzle cap and the nozzle housing, the nozzle tip including a distal end aligned with the delivery outlet and a proximal end aligned and in fluid communication with the flow pathway of a distal end of the conductive insert,
wherein air from the atomizing air chamber is able to flow around the nozzle tip and egress out of the delivery outlet to atomize fluid egressing from the flow pathway.

37. The electrostatic applicator system of Clause 35, wherein the atomizing air chamber includes a volume of at least approximately 900 mm³.

38. The electrostatic applicator system of Clause 35, wherein the nozzle housing includes an air supply port in fluid communication with the atomizing air chamber for atomizing electrostatically charged fluid from the nozzle cap.

39. The electrostatic applicator system of Clause 35, wherein the nozzle cap includes: a plurality of holes on a face of the distal end and selectively arranged around the delivery outlet, the plurality of holes in fluid communication with the atomizing air chamber.

40. The electrostatic applicator system of Clause 39, further including:
a delivery tube at least partially extended from the delivery outlet and in fluid communication with the flow pathway.

41. The electrostatic applicator system of Clause 39, wherein the face of the distal end is curved inwardly or curved outwardly.

42. The electrostatic applicator system of Clause 39, the nozzle cap is configured so that during use, air pumped from the atomizing air chamber through and out through the plurality of holes creates an air curtain around the electrostatically charged fluid contents egressing from the delivery outlet.

43. The electrostatic applicator system of Clause 27, wherein the conductive insert is nested in a cavity of the nozzle housing positioned between the open distal end and the proximal inlet.

44. The electrostatic applicator system of Clause 27, wherein the high voltage connector is a spring extended at least partially through a high voltage port of the nozzle housing and includes a high voltage contact segment extended at least partially through an aperture of the cartridge housing.

45. The electrostatic applicator system of Clause 27, wherein a contact of the high voltage connector to provide a voltage potential of approximately 1 V to approximately 40 kV to the conductive insert.

46. The electrostatic applicator system of Clause 27, wherein a contact of the high voltage connector is in physical contact with the conductive insert to provide a voltage potential of approximately 1 V to approximately 40 kV to fluid in the flow pathway.

47. The electrostatic applicator system of Clause 27, wherein the cartridge housing is a moldable plastic material, wherein the cartridge housing includes a plurality of moldable plastic connectable sections.

48. The electrostatic applicator system of Clause 27, wherein the syringe contains contents including one or more of an antiseptic, a disinfectant solution, an analgesic, an exosome, a biologic, and/or a liquid bandage solution.

49. The electrostatic applicator system of Clause 27, further including: a locking mechanism between a distal end of the syringe and the proximal inlet end of the nozzle housing, the locking mechanism including at least one retainer tab configured to securely connect with the distal end of the syringe and at least one snap connector configured to releasably snap onto the nozzle housing, wherein the locking mechanism comprises a portion of the flow pathway, wherein the portion of the flow pathway of the locking mechanism includes an openable seal.

50. The electrostatic applicator system of Clause 49, wherein the at least one retainer tab is configured prevent the syringe from being removed from the disposable fluid delivery system.

51. The electrostatic applicator system of Clause 27, wherein an outlet diameter of the delivery outlet ranges between approximately a 16-gauge to 25-gauge tube size.

52. A method for operating an electrostatic applicator system, including:
inserting a first disposable cartridge into a chamber housing of the electrostatic applicator system such that:
a voltage contact of a high voltage connector at least partially within a nozzle housing and at least partially extended through a cartridge housing of the first disposable cartridge contacts a voltage contact of the electrostatic applicator system;
an air supply port of the nozzle housing of the first disposable cartridge fluidly connects with an air supply of the electrostatic applicator system; and
a syringe of the first disposable cartridge aligns with a piston of the electrostatic applicator system, the syringe containing a first fluid; and
causing, by an activation input to the electrostatic applicator system, a voltage potential to be delivered by the high voltage connector to a conductive insert of the first disposable cartridge and electrostatically charge fluid contents within a flow pathway.

53. The method of Clause 52, further including:
forming an atomizing air chamber between a nozzle cap and a nozzle housing of the first disposable cartridge to atomize electrostatically charged fluid contents from the flow pathway and egressing through a delivery outlet of the nozzle cap.

54. The method of Clause 53, further including:
directing air, from a pump of the electrostatic applicator system, through an air supply port of the nozzle housing and into the atomizing air chamber; and
causing, by moving the plunger, the electrostatically charged fluid contents from the syringe to delivered through a delivery outlet of the nozzle cap.

55. The method of Clause 52, wherein the first disposable cartridge includes an integrated memory including information related to operational parameters of the first fluid, and wherein inserting the first disposable cartridge into the chamber housing causes the operational parameters to be transmitted to a memory of electrostatic applicator system, the operational parameters including at least one of a speed of the motor, an air intake, and a voltage applied to a flow pathway of the respective disposable cartridge.

What is claimed is:

1. A disposable fluid delivery system for an electrostatic applicator, comprising:
a nozzle cap comprising a distal end comprising a delivery outlet and an open proximal end;
a nozzle housing comprising a proximal inlet end and an open distal end connected to the open proximal end of the nozzle cap;
a conductive insert at least partially positioned between the open distal end and the proximal inlet end and comprising a portion of a flow pathway formed at least partially between the nozzle cap, the nozzle housing, and the conductive insert; and
a high voltage connector extended at least partially through the nozzle housing and electrically connected to the conductive insert and configured to be in electrical communication with a high voltage module and electrostatically charge fluid contents within the flow pathway.

2. The disposable fluid delivery system of claim 1, wherein the high voltage connector comprises a first segment orthogonal relative to a second segment, wherein at least one of the first and second segments comprises an outward flexed contact configured to electrically connect with a high voltage contact of the electrostatic applicator.

3. The disposable fluid delivery system of claim 1, wherein the high voltage connector comprises a first segment angled relative to a second segment, wherein at least one of the first and second segments comprises an outward flexed contact configured to electrically connect with a high voltage contact of the electrostatic applicator.

4. The disposable fluid delivery system of claim 1, further comprising:
a nozzle tip positioned between the nozzle cap and the nozzle housing, the nozzle tip comprising a distal end aligned with the delivery outlet and a proximal end aligned and in fluid communication with the flow pathway of a distal end of the conductive insert;
wherein air from an atomizing air chamber formed between the nozzle housing and the nozzle cap is able to flow around the nozzle tip and egress out of the delivery outlet to atomize fluid egressing from the flow pathway.

5. The disposable fluid delivery system of claim 1, wherein the nozzle housing is hermetically sealed to the nozzle cap forming an atomizing air chamber, and wherein the nozzle housing comprises an air supply port in fluid communication with the atomizing air chamber for atomizing electrostatically charged fluid from the nozzle cap.

6. The disposable fluid delivery system of claim 1, wherein the nozzle cap comprises: a plurality of holes on a face of the distal end and selectively arranged around the delivery outlet, the plurality of holes in fluid communication with an atomizing air chamber formed between the nozzle housing and the nozzle cap.

7. The disposable fluid delivery system of claim 1, wherein the nozzle cap comprises: a plurality of holes on a face of the distal end and selectively arranged around the delivery outlet, the plurality of holes in fluid communication with an atomizing air chamber formed between the nozzle housing and the nozzle cap;
a delivery tube at least partially extended from the delivery outlet and in fluid communication with the flow pathway.

8. The disposable fluid delivery system of claim 1, wherein the nozzle cap comprises: a plurality of holes on a face of the distal end and selectively arranged around the delivery outlet, the plurality of holes in fluid communication with an atomizing air chamber formed between the nozzle housing and the nozzle cap;
wherein the nozzle cap is configured so that during use, air pumped from the atomizing air chamber through and out through the plurality of holes creates an air curtain around the electrostatically charged fluid contents egressing from the delivery outlet.

9. The disposable fluid delivery system of claim 1, further comprising:
a syringe removably connected to the proximal inlet end of the nozzle housing, the syringe comprising a barrel portion and a plunger configured to advance fluids from within the barrel portion and through the flow pathway; and
a cartridge housing at least partially enclosing the nozzle housing, the nozzle cap, the high voltage connector, and the syringe.

10. The disposable fluid delivery system of claim 9, wherein the high voltage connector is a spring extended at least partially through a high voltage port of the nozzle housing and comprises a high voltage contact segment extended at least partially through an aperture of the cartridge housing.

11. The disposable fluid delivery system claim 9, wherein a contact of the high voltage connector is in physical contact with the conductive insert to provide a voltage potential of approximately 1 V to approximately 40 kV to fluid in the flow pathway.

12. The disposable fluid delivery system of claim 9, wherein the syringe contains contents including one or more of an antiseptic, a disinfectant solution, an analgesic, an exosome, a biologic, and/or a liquid bandage solution.

13. The disposable fluid delivery system of claim 9, further comprising: a locking mechanism between a distal end of the syringe and the proximal inlet end of the nozzle housing, the locking mechanism comprising at least one retainer tab configured to securely connect with the distal end of the syringe and at least one snap connector configured to releasably snap onto the nozzle housing, wherein the locking mechanism comprises a portion of the flow pathway.

14. The disposable fluid delivery system of claim 9, further comprising:
a reusable electrostatic applicator including a cartridge chamber sized and shaped to accept the cartridge housing, the reusable electrostatic applicator including:
a high voltage module configured to be in electrical communication with an at least partially moveable voltage contact of the high voltage connector; and
a piston positioned proximate the cartridge chamber and configured to advance the plunger enclosed in the cartridge housing when the cartridge housing is assembled with the cartridge chamber.

15. The disposable fluid delivery system of claim 14, wherein the reusable electrostatic applicator comprises:
a motor configured to move the piston;
one or more processors comprising at least one processing chip; and
memory storing instructions that, when executed by the one or more processors, causes the reusable electrostatic applicator to:
receive an activation signal;
output a control signal to the motor to actuate the piston; and
output a control signal to a switch to provide voltage from the high voltage module to the high voltage connector.

16. An electrostatic applicator system for delivering a treatment solution to a target site, comprising:
a portable reusable electrostatic applicator comprising:
a device housing configured to be handheld;
a motor in the device housing configured to drive a piston;
a voltage source in the device housing;
a high voltage module electrically connected to the voltage source; and
a cartridge chamber; and
a disposable cartridge removably insertable in the cartridge chamber, the disposable cartridge comprising:
a nozzle cap comprising a distal end comprising a delivery outlet and a proximal end;
a nozzle housing comprising a proximal inlet end and an open distal end connected with the open proximal end of the nozzle cap;
a conductive insert between the open distal end and the proximal inlet end and comprising a portion of a flow pathway formed at least partially between the nozzle cap, the nozzle housing, and the conductive insert;
a high voltage connector extended at least partially through the nozzle housing and electrically connected to the conductive insert and in electrical communication with the high voltage module;
a syringe comprising a barrel portion and a plunger configured to advance fluids from within the barrel portion and through the flow pathway; and
a cartridge housing at least partially enclosing the nozzle cap, the nozzle housing, the high voltage connector, and the syringe.

17. The electrostatic applicator system of claim 16, wherein the cartridge chamber comprises a wall comprising a high voltage contact in electrical communication with the high voltage module and an air supply port in fluid communication with a pump positioned in the device housing;
wherein the high voltage connector is in electrical communication with the high voltage contact of the wall; and
wherein the contact of the high voltage connector is in physical contact with an outer surface of the conductive insert to provide a voltage potential of approximately 1 V to approximately 40 kV.

18. A method for operating an electrostatic applicator system, comprising:
inserting a first disposable cartridge into a chamber housing of the electrostatic applicator system such that:
a voltage contact of a high voltage connector at least partially within a nozzle housing and at least partially extended through a cartridge housing of the first disposable cartridge contacts a voltage contact of the electrostatic applicator system;
an air supply port of the nozzle housing of the first disposable cartridge fluidly connects with an air supply of the electrostatic applicator system; and
a syringe of the first disposable cartridge aligns with a piston of the electrostatic applicator system, the syringe containing a first fluid; and
causing, by an activation input to the electrostatic applicator system, a voltage potential to be delivered by the high voltage connector to a conductive insert of the first disposable cartridge and electrostatically charge fluid contents within a flow pathway.

19. The method of claim 18, further comprising:
forming an atomizing air chamber between a nozzle cap and a nozzle housing of the first disposable cartridge to atomize electrostatically charged fluid contents from the flow pathway and egressing through a delivery outlet of the nozzle cap.

20. The method of claim 19, further comprising:
directing air, from a pump of the electrostatic applicator system, through an air supply port of the nozzle housing and into the atomizing air chamber; and
causing, by moving a plunger of the syringe, the electrostatically charged fluid contents from the syringe to delivered through a delivery outlet of the nozzle cap.
